SEQUENCE LISTING (38 sequences)

SEQ ID NO: 1 - DNA, 1220 bp, Saccharomyces cerevisiae
gaaaatactaatacttcttcacacaaaagaacgcagttagacaatcaacaatgaatcctaaatcctctacacctaagattccaagacccaagaacgcatttattctgttcagacagcactaccacaggatcttaatagacgaatggaccgctcaaggtgtggaaataccccataattcaaacatttctaaaattattggtacgaagtggaagggcttacaaccggaagataaggcacactgggaaaatctagcggagaaggagaaactagaacatgaaaggaagtatcctgaatacaaatacaagccggtaagaaagtctaagaagaagcaactacttttgaaggaaatcgagcaacagcagcagcaacaacagaaagaacagcagcagcagaaacagtcacaaccgcaattacaacagccctttaacaacaatatagttcttatgaaaagagcacattctctttcaccatcttcctcggtgtcaagctcgaacagctatcagttccaattgaacaatgatcttaagaggttgcctattccttctgttaatacttctaactatatggtctccagatctttaagtggactacctttgacgcatgataagacggcaagagacctaccacagctgtcatctcaactaaattctattccatattactcagctccacacgacccttcaacgagacatcattacctcaacgtcgctcaagctcaaccaagggctaactcgacccctcaattgccctttatttcatccattatcaacaacagcagtcaaacaccggtaactacaactaccacatccacaacaactgcgacatcttctcctgggaaattctcctcttctccgaactcctctgtactggagaacaacagattaaacagtatcaacaattcaaatcaatatttacctccccctctattaccttctctgcaagattttcaactggatcagtaccagcagctaaagcagatgggaccaacttatattgtcaaaccactgtctcacaccaggaacaatctattgtccacaactacccctacgcatcatcacattcctcatataccaaaccaaaacattcctctacatcaaattataaactcaagcaacactgaggtcaccgctaaaactagcctagtttctccgaaatgattttttttttccatttcttctttccgttatattatattatactatattccctttaactaaaaa

SEQ ID NO: 2 - DNA, 4670 bp, Saccharomyces cerevisiae
tttaactaatgccgaagaattcacccccaaagtgacaaagactatcgcgcaagattcatttgatatattaagctttgatgattccaacactttaattagaaaacaagacacctccgttactataagctttgacgatggtgaaacatgggaaaaagttgaaggcattgaaggcgaaatcacttggatatacatcgaccccttcaatagacatgacagagccgttgcaacggcaatgaatgggtcgtacctttatataaccaatgatcaaggtaagtcatgggagcgtataacgctacctgactccggagaaagtatttcacctcgtgaatgctatatagaaacccatcccttgaataagaactattttcttgcaaagtgcaactattgtgagaaaacagaagtaaacaatgacaatgaagagaattcgggggacgaagagggacaatttgaaatatttaatattacacgttgcacagacaaggtttttgcaagtaatgatggtggaaaatccttttctgagatcaagtcttctctagaaaggaacgaaaatagtcctatcagcatttctgattgtggctttgccaagaccagcaaagattctgaccttgaaagtagtgatacctcaataatctgtctttttcaaaatatgcagcttattatggatgagtttagttctccttacaccgaaagtaaattggtcctaactacggactggggcaaatcactaaaagaatttgaccaatttaaagataaggtcgtcaatggttacaggatattgaaatctcacatggttgtcttaacccagggcgacagatataatgatatgtcttccatggatgtgtgggtatcaaatgatctgtcaaactttaaaatggcttacatgcctactcagttaaggcattctatgcaaggagaaatatatgaggacgctatgggaagaattatcttgcccatgagtagggaaagaagtgatcaagaggaggataagggcatcgtgtctgaaattttaatttccgactcacaagggttaaaattttcccccatcccatggaccgcaaatgaggtgtttggttatattaatttttatcaacctacttacttgaaaggaacgatgattgcctcactttaccctctatctaggcgtcgtaaccgtaaaggaaaagccaaaggagtaaagagtaagggggtaaccaaaatatctgttgataatggcctcacatggacaatgttaaaagttgttgatccagataacgcagactcattcgactgtgatattactgattttgagaattgttcgcttcaaaatatgttttacacacgggagggttccactccaaccgccggaattctaatgacaacaggtattgttggcgatggtagtgtcttcgactggggagatcaaagaacctttatttctagggatggtggcttaacatggaaactcgcctttgattttccttgtttatacgctgttggtgattacggaaatgttattgtggctataccgtataatgcggatgaagacgacgatcctcaatccgaattttattactctttagaccaaggtaaaacttggaccgaatatcagctagaaactactatctacccaaatgaagtaatgaatacaacgcccgacggatctggagctaaatttattctaaatgggtttactttggcgcatatggatggtacaacgaatttcatctatgcaattgatttttcaacagcctttaatgataagacatgcgaagaaaatgatttcgaggattggaatttagctgaggggaagtgtgtcaatggagtcaagtacaagatcagaagaagaaaacaggacgctcagtgcttggtgaagaaagtttttgaagacttacaattatttgagactgcttgtgacaagtgtaccgaggctgattacgaatgcgcgtttgaatttgttagggacgcgaccgggaaatgcgtaccagactacaacctaatcgttctctctgacgtatgtgataagacaaagaaaaaaactgtgcctgtaaaaccattgcaactagttaaaggtgataaatgtaaaaaaccaatgacagtcaaatcagtggatatttcgtgtgagggagttccaaagaagggaacgaatgataaagaaatagtggttacagaaaacaaatttgatttcaagattcaattctatcaatactttgacacagtcaccgacgaatccctcctcatgatcaattcaagaggagaagcttatatatctcatgatggtggacaaacaataaaaaggttcgacagtaatggtgaaacaattattgaagttgtgtttaatccatactacaattcttcagcttatctgtttggttccaaaggtagcattttctctacccatgataggggatactcttttatgactgctaaattgcccgaggctaggcagttaggtatgccattagactttaacgctaaggcacaggatacatttatctattatggtggtaagaattgtgagtcaatcttaagtccggaatgtcatgcggtagcatacctgaccaatgatgggggcgaaacgtttacggaaatgcttgataatgcaattcattgtgagtttgcgggctcacttttcaaatatccgtcaaatgaggatatggttatgtgtcaagtgaaggaaaagtcttcgcagacaagaagcttagtttcttctactgattttttccaggatgataaaaataccgtctttgaaaatattatcggctacttatccactggtggctatatcatcgttgctgttcctcatgagaacaacgaattgagagcatacgtaactatcgatggtactgagtttgccgaggcaaaattcccatatgatgaagatgttgggaagcaagaggcattcactatattagagtctgagaaaggatcgatattcttacatttagcaacaaacttagtaccaggacgcgattttggcaatcttttgaaatccaactcaaatggtacttcttttgtcacgttggagcatgccgttaatagaaacacattcggctatgttgactttgaaaaaattcaaggtctcgaaggcattattctcaccaacatcgtttcaaatagtgacaaggtcgccgagaataaagaagacaaacaattgaagacgaagatcacctttaatgaaggttcagattggaactttttgaaacctccgaagagggattcagaaggaaaaaagttttcttgcagctccaaatcactggatgagtgttcattgcacttacatggctatactgaacgtaaggatattagagatacatattcttccggttctgcattaggaatgatgttcggcgttggcaacgttggtcctaaccttttaccatataaagaatgttccaccttcttcaccaccgatggtggcgaaacgtgggctgaagttaagaagactcctcaccaatgggaatacggtgaccacggtgggattttagttttagttcctgaaaactcagaaactgattctatttcctattctaccgattttggtaaaacatggaaagattataaattctgcgctgataaggttttagtaaaggatataaccactgttcccagggattctgctttgagatttttgctgtttggagaggcagcagatattggaggcagctcatttagaacgtacacaattgattttagaaacatcttcgaaagacaatgtgatttcgacatcactggtaaggaaagcgcagattataaatactctcctctgggttccaaaagcaattgcctatttggtcaccaaaccgagtttttacgtaaaaccgatgaaaattgttttattgggaatattccactttctgaattttcaagaaatatcaaaaactgttcttgtacaagacaagatttcgagtgtgattacaacttttacaaagctaacgatggtacttgtaaattagtcaaaggactaagcccagcaaatgctgcagacgtttgtaaaaaagagccagatttaatcgaatattttgaatcgtcaggctacagaaagatccctctatcaacctgtgagggtggcctgaaattggatgctccctcatcaccacatgcttgcccaggaaaagaaaaagaattcaaggaaaagtactcagtaagtgccggtccctttgcatttattttcatttcaattcttttaataattttctttgccgcatggtttgtatatgacagaggtatcagaagaaatgggggatttgcaaggtttggagaaattaggctaggtgacgatggtttaatagaaaacaataatactgacagagttgtcaataacattgtgaaatcaggattttacgttttctcaaatatcgggtctcttttacagcacacaaaaactaatatagcgcatgctatctccaaaattagaggaaggtttggaaacagaacaggtccaagctactcatccctgatccatgatcaatttttggatgaagcagatgacctgcttgctggccacgatgaagacgccaatgacttatccagtttcatggatcagggtagtaattttgaaatcgaagaagatgatgttccaacacttgaagaagagcatacatcatatacagatcaacctacgaccaccgatgttccagatacattaccagaaggaaatgaggaaaacatcgacaggcctgattctacagcgc

SEQ ID NO: 3 - DNA, 1095 bp, Saccharomyces cerevisiae
aatataacacagcatatatacacacacacacataaaataaccgcaaaaatggaggacagtagcaatacaatcataccctcacccactgacgtgggggcgctagcaaacagaatcaactttcaaaccagagatgcccacaataaaatcaataccttcatgggcataaagatggccatcgccatgagacatggctttatatacagacagggtattctggcgtactattatgtgttcgatgccatcgagcaagagatagatcgcctactgaatgaccccgtaacggaggaggagctgcaaacttcgaccattctgaagcagttttggctcgaagattttagaagatctacgcagatctataaggacctgaagctgctatactcaaacacgtttaaaagcacagaatcattaaacgaattcctggctacgttccagaagccaccgctactacagcagtttatcaataacatccacgaaaacatacacaaggagccatgcaccattctttcttactgtcacgttctgtacttggcgcttttcgccggcggcaagctaatacgatcgaatttgtacagaagactggggctcttccccaacttcgagaagctatcacagaaggaactggtcaaaaagggcacaaacttcttcaccttcagcgatctgggtcccactgaagaaacacgcttgaaatgggaatacaagaagaactatgagctggccaccaggacggaattgaccgaagcacaaaagttgcagatcattagcgtcgcagaaggcatttttgattggaacttcaacatcgttgcagaaattggagagttgaatcgtcgcgagttaatgggcaagttcagcttcaagtgtattacgtacttgtacgaagaatggatgttcaacaaggattctgctactagaagagcactccacacggtcatgctgctggtgctttctattatcgcgatctgggttctttacttcttggtaaagagttttcttagcatagtataacgtataaaaaaaaaagtctaaatacaaacataatatatacatgaaataatatcaaaatatgaagaatagggaaggaaaaacagcagagaaaaa

SEQ ID NO: 4 - DNA, 1181 bp, Saccharomyces cerevisiae
gccaaccaagttgctgcaaaagtccacaaggctaaaatttataaacacgagttgtccgatgagatgaaagaagtcactttcgagcaacatttagctcatttaggccaaaagtacttgactcaatttgagaaagctaaccccgaagttgttttttctagggagcatcctttcttcactgaaggtggtcacgatgttccattgacaaattacttgaacgcacaatattacactgacattactttgggtactccacctcaaaacttcaaggttattttggatactggttcttcaaacctttgggttccaagtaacgaatgtggttccttggcttgtttcctacattctaaatacgatcatgaagcttcatcaagctacaaagctaatggtactgaatttgccattcaatatggtactggttctttggaaggttacatttctcaagacactttgtccatcggggatttgaccattccaaaacaagacttcgctgaggctaccagcgagccgggcttaacatttgcatttggcaagttcgatggtattttgggtttgggttacgataccatttctgttgataaggtggtccctccattttacaacgccattcaacaagatttgttggacgaaaagagatttgccttttatttgggagacacttcaaaggatactgaaaatggcggtgaagccacctttggtggtattgacgagtctaagttcaagggcgatatcacttggttacctgttcgtcgtaaggcttactgggaagtcaagtttgaaggtatcggtttaggcgacgagtacgccgaattggagagccatggtgccgccatcgatactggtacttctttgattaccttgccatcaggattagctgaaatgattaatgctgaaattggggccaagaagggttggaccggtcaatatactctagactgtaacaccagagacaatctacctgatctaattttcaacttcaatggctacaacttcactattgggccatacgattacacgcttgaagtttcaggctcctgtatctctgcaattacaccaatggatttcccagaacctgttggcccactggccatcgttggtgatgccttcttgcgtaaatactattctatttacgatttgggcaacaatgcggttggtttggccaaagcaatttgagctaaactttt

SEQ ID NO: 5 - DNA, 309 bp, Saccharomyces cerevisiae
atggctttattaaaagccaataaggatttaatttcagcaggtttgaaagaatttagtgttcttctaaatcaacaagtttttaatgatcctttggtctctgaagaagatatggttactgttgttgaagattggatgaatttttatattaattattataaacaacaagttactggtgaacctcaagaaagagataaagctctacaagaactaaggcaagaactaaatactctagctaatccatttttggctaaatatagagattttctaaaatctcatgaattgccatctcatccaccaccatcttcttag

SEQ ID NO: 6 - protein, 102 aa, Saccharomyces cerevisiae
MALLKANKDLISAGLKEFSVLLNQQVFNDPLVSEEDMVTVVEDWMNFYINYYKQQVTGEPQERDKALQELRQELNTLANPFLAKYRDFLKSHELPSHPPPSS

SEQ ID NO: 7 - DNA, 984 bp, Saccharomyces cerevisiae
atgggccctgaaactctacatattggtgggagaaaatcgaaattggcggtaatacaatccaaccatgttttaaaactgatcgaagaaaagtatccggactacgactgcaaggttttcactttgcaaactcttggtgaccagattcaattcaaacctttgtactcatttggcggtaaagctttatggacaaaggagttggaagaccatctttaccatgacgatccctcaaagaagcttgacttgatcgttcattctctgaaggacatgcccactttactaccagagggtttcgagctggggggtatcactaagcgggtcgatccaacagattgtcttgtcatgcccttttactctgcttataagtctctggatgaccttccagacggggggattgtgggaacctcatccgtgagaagatctgctcagctaaaaagaaaatacccacatttgaaatttgaaagtgtcagaggaaatatacaaactagattacaaaaactagacgacccaaaatctccgtaccaatgcatcatcttggcgtctgctgggttgatgcgtatggggttggaaaacagaattacgcagcgattccattcggatacaatgtaccatgcagttggacaaggcgccctgggtatagaaattagaaagggtgacaccaagatgatgaagattcttgacgaaatttgcgatctaaatgcaactatatgttgcctttcggagcgtgctttgatgagaactttagaggggggttgttccgttcctattggtgtggaatctaaatacaatgaagagactaaaaaattactattaaaggccattgtagttgacgttgaaggcacagaagcagtagaagacgaaattgaaatgctaatagaaaatgttaaagaagattccatggcgtgtggtaagatactagctgaaagaatgattgccgatggcgcaaagaaaattctggatgaaattaatttagacagaatcaaatga

SEQ ID NO: 8 - protein, 327 aa, Saccharomyces cerevisiae
MGPETLHIGGRKSKLAVIQSNHVLKLIEEKYPDYDCKVFTLQTLGDQIQFKPLYSFGGKALWTKELEDHLYHDDPSKKLDLIVHSLKDMPTLLPEGFELGGITKRVDPTDCLVMPFYSAYKSLDDLPDGGIVGTSSVRRSAQLKRKYPHLKFESVRGNIQTRLQKLDDPKSPYQCIILASAGLMRMGLENRITQRFHSDTMYHAVGQGALGIEIRKGDTKMMKILDEICDLNATICCLSERALMRTLEGGCSVPIGVESKYNEETKKLLLKAIVVDVEGTEAVEDEIEMLIENVKEDSMACGKILAERMIADGAKKILDEINLDRIK

SEQ ID NO: 9 - DNA, 354 bp, Homo sapiens
atggtcttgtctcctgccgataaaactaatgtaaaagctgcctggggtaaagtaggtgcccacgccggtgaatatcacggttcagcacaagtcaagggtcatggtaaaaaggtagccgacgctttgaccaatgcagttgcccacgtcgatgacatgccaaacgctttgtccgcattaagtgatttgcatgctcacaagttgagagtagaccctgttaacttcaagttgttgtcccattgtttgttagttaccttagctgcacacttgccagccgaattcactcctgctgtccatgcttctttagacaagtttttagcatccgtttccaccgttttgacctccaagtatagataa

SEQ ID NO: 10 - DNA, 444 bp, Homo sapiens
atggtacatttgactccagaagaaaagtccgccgtaacagcattatggggtaaagtaaacgtagacgaagttggtggtgaagccttaggtagattgttagttgtctatccatggacccaaagatttttcgaatcttttggtgacttgtctactccagacgccgtaatgggtaatcctaaagttaaggctcatggtaaaaaggttttgggtgctttttccgatggtttggcacacttagacaacttgaaaggtactttcgcaacattgtctgaattgcattgtgataagttacacgttgatcctgaaaacttcagattgttgggtaacgtattagtttgcgtcttggctcatcactttggtaaagaattcactccacctgttcaagcagcctatcaaaaggttgtcgcaggtgttgctaacgcattggcacataagtatcactga

SEQ ID NO: 11 - protein, 117 aa, Homo sapiens
MVLSPADKTNVKAAWGKVGAHAGEYHGSAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 12 - protein, 147 aa, Homo sapiens
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 13 - DNA, 1173 bp, Artificial Sequence
  Synthetic sequence encoding hemoglobin secreted from modified yeast cell, delta rox1 delta vps10 delta hmx1 delta pep4/HEM3+alpha leader-Hbfusion
atgagatttccatctatttttactgctgttttgtttgctgcttcttctgctttggctgctccagttaatactactactgaagatgaaactgctcaaattccagctgaagctgttattggttattctgatttggagggtgactttgatgttgctgttttgccattttctaactctactaacaacggtttgctattcatcaacactactatcgcttctatcgctgctaaagaagaaggtgtttctttggataaaagagaagaaggtgaaccaaaagtcttatctcctgccgataaaacaaatgtaaaagctgcctggggtaaagtaggtgctcatgccggtgaatacggtgctgaagccttagaaagaatgtttttgtctttcccaactacaaagacctacttccctcatttcgatttgtctcacggttcagcacaagtcaagggtcatggtaaaaaggtagccgacgctttgacaaatgcagtcgcccacgtagatgacatgccaaacgccttgtccgctttaagtgatttgcatgctcacaaattaagagttgaccctgtcaacttcaagttgttgtcccattgtttgttggttactttggctgcacacttgccagccgaattcactcctgcagttcatgcctccttagataaattcttggcttccgtaagtacagttttgaccagtaagtatagaggtggttctggtggttcaggtggttccggtggtagtggtcattttactgaagaagataaagctaccatcacttcattatggggtaaagtaaacgttgaagacgcaggtggtgaaaccttgggtagattgttagttgtctacccatggactcaaagatttttcgattctttcggtaatttgtcttctgcttcagcaattatgggtaaccctaaagttaaggctcatggtaaaaaggtcttaacttctttgggtgacgctattaaacacttagatgacttgaagggtacatttgcccaattatcagaattgcattgcgataaattgcacgttgacccagaaaatttcaagttgttgggtaacgtcttagtaacagttttggcaatccatttcggtaaagaattcacccctgaagttcaagcaagttggcaaaagatggtcacaggtgtcgcctccgcattgagttccagatatcattga

SEQ ID NO: 14 - protein, 390 aa, Artificial Sequence
  Synthetic polypeptide sequence of hemoglobin secreted from modified yeast cell, delta rox1 delta vps10 delta hmx1 delta pep4/HEM3+alpha leader-Hbfusion
MRFPSIFTAVLFAASSALAAPVNTTTEDETAQIPAEAVIGYSDLEGDFDVAVLPFSNSTNNGLLFINTTIASIAAKEEGVSLDKREEGEPKVLSPADKTNVKAAWGKVGAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYRGGSGGSGGSGGSGHFTEEDKATITSLWGKVNVEDAGGETLGRLLVVYPWTQRFFDSFGNLSSASAIMGNPKVKAHGKKVLTSLGDAIKHLDDLKGTFAQLSELHCDKLHVDPENFKLLGNVLVTVLAIHFGKEFTPEVQASWQKMVTGVASALSSRYH

SEQ ID NO: 15 - DNA, 66 bp, Artificial Sequence
  Synthetic oligonucleotide
catactctcttgcttagtctaaggaggagctatttaacagtgcacgacaacccttaattaccgttc

SEQ ID NO: 16 - DNA, 58 bp, Artificial Sequence
  Synthetic oligonucleotide
caacctattaatttcccctcgtcaaaaataaggttatcaagtgagaaatcaccatgag

SEQ ID NO: 17 - DNA, 55 bp, Artificial Sequence
  Synthetic oligonucleotide
ggcaaaacagcattccaggtattagaagaatatcctgattcaggtgaaaatattg

SEQ ID NO: 18 - DNA, 74 bp, Artificial Sequence
  Synthetic oligonucleotide
agctatctcagggtagataatagggctgtaaaaacctctcacatgggatctgatataccgttcgtatagcatac

SEQ ID NO: 19 - DNA, 66 bp, Artificial Sequence
  Synthetic oligonucleotide
attacttcattttgtctattctctttgggccttacttctcattccgacaacccttaattaccgttc

SEQ ID NO: 20 - DNA, 75 bp, Artificial Sequence
  Synthetic oligonucleotide
tatctactctatgtaaagtaatctctctactggttttcgttagatgggatctgatataccgttcgtatagcatac

SEQ ID NO: 21 - DNA, 68 bp, Artificial Sequence
  Synthetic oligonucleotide
atgttcagcttgaaagcattattgccattggccttgttgttggtcaggacaacccttaattaccgttc

SEQ ID NO: 22 - DNA, 76 bp, Artificial Sequence
  Synthetic oligonucleotide
tataaaagctctctagatggcagaaaaggatagggcggagaagtaagggatctgatataccgttcgtatagcatac

SEQ ID NO: 23 - DNA, 36 bp, Artificial Sequence
  Synthetic oligonucleotide
cctggatccatggctttattaaaagccaataaggat

SEQ ID NO: 24 - DNA, 35 bp, Artificial Sequence
  Synthetic oligonucleotide
tttctcgagctaagaagatggtggtggatgagatg

SEQ ID NO: 25 - DNA, 57 bp, Artificial Sequence
  Synthetic oligonucleotide
ctactttttacaacaaatataaaacaaggatccatgcgaatccccgggttaattaac

SEQ ID NO: 26 - DNA, 47 bp, Artificial Sequence
  Synthetic oligonucleotide
gttttatcggcaggagataagactttgtatagttcatccatgccatg

SEQ ID NO: 27 - DNA, 47 bp, Artificial Sequence
  Synthetic oligonucleotide
catggcatggatgaactatacaaagtcttatctcctgccgataaaac

SEQ ID NO: 28 - DNA, 38 bp, Artificial Sequence
  Synthetic oligonucleotide
gcggtaccaagcttactcgagtcaatgatatctggaac

SEQ ID NO: 29 - DNA, 29 bp, Artificial Sequence
  Synthetic oligonucleotide
aaaggatccatgcggtgagcaagggcgag

SEQ ID NO: 30 - DNA, 31 bp, Artificial Sequence
  Synthetic oligonucleotide
aaactcgagtcattccgtcgccccccggtag

SEQ ID NO: 31 - DNA, 44 bp, Artificial Sequence
  Synthetic oligonucleotide
cacatacataaactaaaaggtaccaacaaaatgagatttccatc

SEQ ID NO: 32 - DNA, 35 bp, Artificial Sequence
  Synthetic oligonucleotide
tcggcaggagataagacttttggttcaccttcttc

SEQ ID NO: 33 - DNA, 35 bp, Artificial Sequence
  Synthetic oligonucleotide
gaagaaggtgaaccaaaagtcttatctcctgccga

SEQ ID NO: 34 - DNA, 38 bp, Artificial Sequence
  Synthetic oligonucleotide
gtttctagactcgaggctagctcaatgatatctggaac

SEQ ID NO: 35 - DNA, 38 bp, Artificial Sequence
  Synthetic oligonucleotide
agcaaccgttggcatggatccctaggaattggagcgac

SEQ ID NO: 36 - DNA, 38 bp, Artificial Sequence
  Synthetic oligonucleotide
gatctggccggccggatccccgcacacaccatagcttc

SEQ ID NO: 37 - DNA, 43 bp, Artificial Sequence
  Synthetic oligonucleotide
caacaaatataaaacaaggatccaacaaaatgagatttccatc

SEQ ID NO: 38 - DNA, 38 bp, Artificial Sequence; identical to SEQ ID NO: 28
  Synthetic oligonucleotide

(19) United States
(12) Patent Application Publication
(10) Pub. No.: US 2025/0388852 A1
(43) Pub. Date: Dec. 25, 2025

(54) A GENETICALLY MODIFIED YEAST CELL FOR HEMOGLOBINS PRODUCTION

(71) Applicant: CHRYSEA LIMITED, Dublin (IE)

(72) Inventors: Olena ISHCHUK, Göteborg (SE); Dina PETRANOVIC NIELSEN, Hellerup (DK)

(21) Appl. No.: 18/250,998

(22) PCT Filed: Nov. 11, 2021
(86) PCT No.: PCT/SE2021/051128

§ 371 Date: Apr. 28, 2023

(30) Foreign Application Priority Data
Nov. 11, 2020 (SE) 2051317-2

(51) Int. Cl. C12N 1/18; C07K 14/805; C12N 9/02; C12N 9/10; C12R 1/78; C12R 1/84; C12R 1/865

(52) U.S. Cl. CPC C12N 1/18; C07K 14/805; C12N 9/0071; C12N 9/1085; C12Y 205/01061; C12N 2510/00; C12R 2001/78; C12R 2001/84; C12R 2001/865

(57) ABSTRACT

A genetically modified yeast cell, wherein the yeast cell comprises a genetic modification comprising overexpression of yeast gene encoding porphobilinogen deaminase (HEM3), the HEM3 gene having at least 80% identity with SEQ ID No. 7. The genome of the modified yeast cell further comprises one or more genetic modifications in one or more genes selected from: genes coding for heme-dependent repressor of hypoxic genes (ROX1), genes coding for heme oxygenase (HMX1), genes coding for a receptor for vacuolar proteases (VPS10), and genes coding for vacuolar proteinase (PEP4), the one or more genetic modifications being such that expression of a polypeptide from such a gene is reduced or disrupted or the polypeptide expressed is non-functional.

### STATEMENT REGARDING ELECTRONIC FILING OF A SEQUENCE LISTING

A Sequence Listing in ASCII text format, submitted under 37 C.F.R. § 1.821, entitled 9737-103_ST25.txt, 31,496 bytes in size, generated on May 30, 2025, and filed electronically, is provided in lieu of a paper copy. This Sequence Listing is hereby incorporated herein by reference into the specification for its disclosures.

### TECHNICAL FIELD

The present invention relates to a genetically modified yeast cell, which may be used in production of human and non-human hemoglobins.

### BACKGROUND ART

Hemoglobin (Hb) is a major blood protein in erythrocytes (red blood cells, RBC) in blood circulation, whose main function is to carry oxygen from lungs to tissues. RBCs contain as much as 98% of Hb with respect to the total soluble protein content. Hemoglobin is a tetrameric cofactor-containing protein, and in adult humans it is composed of two α- and two β-globin subunits (α₂β₂), encoded by genes HBA and HBB, correspondingly. Each hemoglobin subunit carries one non-covalently bound heme b (protoporphyrin IX) group with a ferrous iron atom ligated by the four nitrogens at the center of the porphyrin ring. The iron atom is an active site for oxygen binding, while the organic component of the protein contributes to regulation, for example ensures the reversibility of oxygen binding.

With an increasing need for oxygen carriers for transfusion, production of human hemoglobin (Hb) from sustainable sources is increasingly in demand. Microbial production is one of the attractive options, as it may provide a cheap, safe, and reliable source of this protein. However, the production of cofactor-containing proteins, including Hb, is challenging since the loss of the cofactor is usually associated with loss of activity. The research on recombinant production of hemoglobin was ongoing during the last four decades by using different production hosts covering almost all kingdoms: bacteria, yeast, animals, and plants.

To obtain the stochiometric amounts of α- and β-globins, which is important for Hb folding, the human globin genes were expressed in a single operon in E. coli (Hoffman S J, Looker D L, Roehrich J M et al. Expression of fully functional tetrameric human hemoglobin in Escherichia coli. Proc Natl Acad Sci USA. 1990. 87 (21): 8521-8525.). Site-directed mutagenesis was used to improve the solubility of Hb upon the expression in bacteria (Weickert M J, Pagratis M, Glascock C B et al. A mutation that improves soluble recombinant hemoglobin accumulation in Escherichia coli in heme excess. Appl Environ Microbiol. 1999. 65 (2): 640-647.). In E. coli the co-expression of erythroid human α-hemoglobin stabilizing protein (AHSP) was proven successful for increasing Hb yields (Vasseur-Godbillon C, Hamdane D, Marden M C et al. High-yield expression in Escherichia coli of soluble human alpha-hemoglobin complexed with its molecular chaperone. Protein Eng Des Sel. 2006. 19 (3): 91-97.).

Yeast has been used for food and beverages for thousands of years and is generally regarded as safe. Yeast Saccharomyces cerevisiae is a traditional model organism for academic and industrial research and applications. The advances of genetic engineering, genomics, systems and synthetic biology methods and tools made it one of the preferred cell factories for a wide array of chemicals, fuels, flavors, industrial enzymes and pharmaceutical products. Production of heme-containing proteins is in demand for the development of blood and meat substitutes. The main target proteins for these markets are hemoglobins, hence making the production dependent on heme availability. While for blood substitutes researched bovine and human hemoglobins, food applications are interested in hemoglobins of plant origin, for example, legume hemoglobin (Fraser R Z, Shiut M, Agrawal P, et al. Safety Evaluation of Soy Leghemoglobin Protein Preparation Derived From Pichia pastoris, Intended for Use as a Flavor Catalyst in Plant-Based Meat. Int J Toxicol. 2018; 37 (3): 241-262; and Fraser R, O'Reilly Brown P, et al. Methods and compositions for affecting the flavor and aroma profile of consumables. US 2015/0351435 A1. Dec. 10, 2015. Impossible Foods Inc., Redwood City, CA (US).)

S. cerevisiae produces heme endogenously in a complex pathway involving cytosol and mitochondria compartments, and this process is strictly regulated by a carbon source, oxygen, and heme availability (Zhang L, Hach A. Molecular mechanism of heme signaling in yeast: the transcriptional activator Hap1 serves as the key mediator. Cell Mol Life Sci. 1999. 56 (5-6): 415-426; and Hoffman M, Góra M, Rytka J. Identification of rate-limiting steps in yeast heme biosynthesis. Biochem Biophys Res Commun. 2003. 310 (4): 1247-1253.). Heme biosynthesis initiates inside the mitochondria with the condensation of two precursors, succinyl-Co A and glycine. The 5-aminolevulinic acid (5-ALA), which is the product of this reaction, is then transported into the cytosol, where it is converted into coproporphyrinogen Ill by the next series of enzymatic reactions. Further oxidative decarboxylation and oxidation steps in mitochondria yield protoporphyrin IX. The insertion of iron by a mitochondrial ferrochelatase finalizes the process.

The expression of recombinant hemoglobin in yeast has been significantly improved by the strategies based on enhancing the endogenous heme biosynthesis, balancing α- and β-globin gene expression, and engineering of cell oxygen sensing (Liu L, Martínez J L, Liu Z et al. Balanced globin protein expression and heme biosynthesis improve production of human hemoglobin in Saccharomyces cerevisiae. Metab Eng. 2014. 21:9-16; and Martínez J L, Liu L, Petranovic D et al. Engineering the oxygen sensing regulation results in an enhanced recombinant human hemoglobin production by Saccharomyces cerevisiae. Biotechnol Bioeng. 2015. 112 (1): 181-188.). The heme biosynthesis capacity was increased by the overexpression of rate-limiting enzymes of the pathway (Hoffman M, Góra M, Rytka J. Identification of rate-limiting steps in yeast heme biosynthesis. Biochem Biophys Res Commun. 2003. 310 (4): 1247-1253; and Liu L, Martínez J L, Liu Z et al. Balanced globin protein expression and heme biosynthesis improve production of human hemoglobin in Saccharomyces cerevisiae. Metab Eng. 2014. 21:9-16.). As an example, the overexpression of HEM3 gene (encoding porphobilinogen deaminase) on multi-copy plasmid results in up to 4-fold increase in intracellular free heme (Liu L, Martínez J L, Liu Z et al. Balanced globin protein expression and heme biosynthesis improve production of human hemoglobin in Saccharomyces cerevisiae. Metab Eng. 2014. 21:9-16.). Because environmental oxygen's levels regulates the intracellular level of heme, the engineering of oxygen sensing by deletion of the HAP1 gene, coding for a transcription factor involved in the regulation of cellular respiration, was successful to improve the hemoglobin production further, up to 7% of the total cell soluble protein content (Martínez J L, Liu L, Petranovic D et al. Engineering the oxygen sensing regulation results in an enhanced recombinant human hemoglobin production by Saccharomyces cerevisiae. Biotechnol Bioeng. 2015. 112 (1): 181-188.).

In view of the state of the art there is, hence, a need for an improved hemoglobin production method with higher yield from cheap substrates, for example, comprising glucose.

### SUMMARY

It is an object of the present disclosure to provide a genetically modified yeast cell. The genetically modified yeast cell being suitable for the production of human hemoglobin and non-human hemoglobins. The modified yeast cell providing an improved hemoglobin yield compared to state-of-the-art methods.

The invention is defined by the appended independent claims. Non-limiting embodiments emerge from the dependent claims, the appended drawings, and the following description.

According to a first aspect there is provided a genetically modified yeast cell, wherein the yeast cell comprises a genetic modification comprising overexpression of yeast gene encoding porphobilinogen deaminase (HEM3), the HEM3 gene having at least 80% identity with SEQ ID No. 7. The genome of the modified yeast cell further comprises one or more genetic modifications in one or more genes selected from: genes coding for heme-dependent repressor of hypoxic genes (ROX1), genes coding for heme oxygenase (HMX1), genes coding for a receptor for vacuolar proteases (VPS10), and genes coding for vacuolar proteinase (PEP4), the one or more genetic modifications being such that expression of a polypeptide from such a gene is reduced or disrupted or the polypeptide expressed in non-functional.

The yeast cell comprises one or more genetic modifications in one or more genes, for example in the ROX1 and/or HMX1 genes, such that the one or more genetic modifications is such that expression of a polypeptide from such a gene is reduced or disrupted or the polypeptide expressed is non-functional. This may be accomplished by elimination (deletion) of the entire coding region of the gene, or the gene or its promoter and/or terminator region is modified (such as by deletion, insertion, or mutation) such that the gene no longer produces a partially or fully functional polypeptide, e.g. the activity of the protein is reduced or eliminated. The modification can be accomplished by genetic engineering methods, forced evolution or mutagenesis, and/or selection or screening.

The yeast cell comprises a genetic modification comprising overexpression of yeast gene encoding porphobilinogen deaminase (HEM3), the HEM3 gene having at least 80%, or at least 90%, or at least 95%, or 100% identity with SEQ ID No. 7. The gene being HEM3 may be located on a yeast plasmid (such as plYC04).

The method of overexpression can be achieved: either by introducing 1, 2, 3, 4 or more copies of genes encoding porphobilinogen deaminase (HEM3) into the yeast genome or by multi-copy of plasmids; or by substituting a native promoter of HEM3 gene by a strong constitutive promoter (such as promoters of genes TEF1 or PGK1); or by modifying the native promoter of HEM3 gene to increase the transcription of HEM3 gene, or by modifying the native terminator of the HEM3 gene to increase the half-life of the HEM3 gene transcript; or by modifying the proteins involved in regulation of HEM3 gene translation (either repressors or activators) to result in increased level of Hem3 polypeptide. The Hem3 polypeptide may be at least 95% identical with SEQ ID No. 8, or have Hem3 activity in yeast.

This genetically modified yeast cell is for example suitable for production of human hemoglobins or non-human hemoglobins when genes encoding for such hemoglobins have been introduced in the genome of the modified yeast cell, possibly also overexpressing such hemoglobin genes. The modified yeast cell providing an improved hemoglobin yield compared to state-of-the-art methods.

The yeast genome of the modified yeast cell may comprise one or more genetic modifications in the genes coding for a heme-dependent repressor of hypoxic genes (ROX1).

The ROX1 gene is located on chromosome XVI of the yeast genome, from position 679643 to 680862, SEQ ID No. 1. By one or more genetic modifications in the gene coding for ROX1, inhibition of the HEM13 gene (encoding coproporphyrinogen Ill oxidase) by heme-dependent repressor Rox1 can be eliminated.

The genetic modification may comprise deletion of the Open Reading Frame (ORF) of the ROX1 gene. As alternative mutations, partial deletions of the ROX1 ORF could be performed, which may result in production of truncated Rox1 polypeptides with no Rox1 activity or which terminate the translation of the Rox1 polypeptide. Any genetic modifications that result in non-functional Rox1, for example, partial gene deletions or insertions that disrupt ROX1 open reading frame are possible.

The yeast genome of the modified yeast cell may comprise one or more genetic modifications in the genes coding for a receptor for vacuolar proteases (VPS10).

The VPS10 gene is located on chromosome Il of the yeast genome, from position 191533 to 186864, SEQ ID No. 2. By the one or more genetic modifications of the VPS10 gene, there may be an improved porphyrins and hemoglobin production when using the genetically modified yeast cell for hemoglobin production, and also a reduced formation of the degradation product of hemoglobin (bilirubin). The targeting of hemoglobin to the vacuoles for the protein degradation may be suppressed by genetically modifying the VPS10 gene (sorting receptor of vacuolar hydrolases).

The modification may comprise deletion of the VPS10 ORF. Alternative mutations may comprise modifications that result in no translation of the Vps10 polypeptide. As alternative mutations, partial deletions of VPS10 ORF could be performed, which result in production of truncated Vps10 polypeptide with no Vps10 activity. Any kind of mutations: insertions, deletions, nucleotide substitutions, which cause the disruption of VPS10 open reading frame or no Vps10 polypeptide produced or inactive Vps10 polypeptide are possible.

The yeast genome of the modified yeast cell may comprise one or more genetic modifications in the genes coding for heme oxygenase (HMX1).

The HMX1 gene is located on chromosome XII of the genome, from position 553725 to 552631, SEQ ID No. 3.

By the one or more genetic modifications of the HMX1 gene there may be an improved porphyrins and hemoglobin production, when using the genetically modified yeast cell for hemoglobin production, and also a reduced formation of degradation product of hemoglobin (bilirubin). The HMX1 gene encodes heme oxygenase, responsible for specific heme cleavage.

The modification may comprise deletion of the HMX1 ORF. Alternative mutations may comprise partial deletions of the HMX1 ORF, which result in production of truncated Hmx1 polypeptide with no Hmx1 activity. The alternative mutations may be any kind of mutations: insertions, deletions, nucleotide substitutions, which cause the disruption of the HMX1 open reading frame resulting in no Hmx1 polypeptide produced or production of inactive Hmx1 polypeptide.

The modified yeast cell may comprise one or more genetic modifications in the genes coding for vacuolar proteinase A (PEP4).

The PEP4 gene is located on chromosome XVI of the genome, from position 260883 to 259703, SEQ ID No. 4.

By the one or more genetic modifications of the PEP4 gene there may be an improved porphyrins and hemoglobin production when using the genetically modified yeast cell for hemoglobin production. The targeting of hemoglobin to the vacuoles for the protein degradation may be suppressed by deleting the PEP4 (vacuolar proteinase A) gene.

The modification may comprise deletion of the PEP4 ORF. Alternative mutations may comprise partial deletions of the PEP4 ORF, which may result in production of truncated Pep4 polypeptide with no Pep4 activity. The alternative mutations may be any kind of mutations: insertions, deletions, nucleotide substitutions, which cause the disruption of the PEP4 open reading frame resulting in no Pep4 polypeptide produced or production of inactive Pep4 polypeptide.

A genetically modified yeast cell comprising several or all of the genetic modifications selected from ROX1 genes, HMX1 genes, VPS10 genes, and PEP4 genes show, when used for hemoglobin production, an improved hemoglobin yield compared to a genetically modified yeast cell comprising fewer of these genetic modifications. A genetically modified yeast cell comprising at least one of the genetic modifications selected from ROX1 genes, HMX1 genes, VPS10 genes, and PEP4 genes show, when used for hemoglobin production, an improved hemoglobin yield compared to a yeast cell comprising none of these genetic modifications.

The genetically modified yeast cell may comprise a human gene encoding erythroid molecular chaperone (AHSP), the AHSP gene having at least 80%, at least 90%, at least 95% or at least 100% identity with SEQ ID No. 5, and wherein the AHSP gene is overexpressed. The AHSP produced being at least 95% identical with SEQ ID No. 6, or the polypeptide produced having AHSP activity in yeast.

The AHSP gene may be located on a yeast plasmid (such as plYC04).

Overexpression of alpha-hemoglobin-stabilizing protein (AHSP) may result in as much as a 58% increase in hemoglobin production when the modified cell is used for hemoglobin production compared to a genetically modified yeast cell strain Δrox1Δvps10Δhmx1Δpep4 (i.e. comprising genetic modifications in the ROX1 genes, HMX1 genes, VPS10 genes, and PEP4 genes) without such ASHP overexpression.

The method of overexpression can be achieved: either by introducing 1, 2, 3, 4 or more copies of genes encoding erythroid molecular chaperone (AHSP) into the yeast genome or by multi-copy of the plasmid; or by substituting a native promoter of the AHSP gene by a strong constitutive promoter (such as promoters of genes TEF1 or PGK1); or by modifying the native promoter of the AHSP gene to increase the transcription of the AHSP gene or by modifying the native terminator of the AHSP gene to increase the half-life of the AHSP gene transcript; or by modifying the proteins involved in regulation of AHSP gene translation (either repressors or activators) to result in increased level of AHSP polypeptide.

In one experiment, a genetically modified yeast cell as described above comprising modifications in all of the ROX1 genes, HMX1 genes, VPS10 genes, and PEP4 genes, and overexpression of AHSP, and used for hemoglobin production, showed a 1.9 times higher production of total porphyrins comprising human hemoglobin, compared to a yeast cell (such as WT/H3/ααβ strain) that expresses human hemoglobin but does not have the described modifications (deletions of ROX1, HMX1, VPS10, PEP4 and overexpression of AHSP).

The genetically modified yeast cell described above may comprise genes coding for human hemoglobin or genes coding for non-human hemoglobins, the non-human hemoglobins containing heme as a cofactor and a globin part that reversibly binds gaseous ligands.

The genes coding for human hemoglobin subunit alpha, HBA, SEQ ID No. 9, and the hemoglobin subunit beta, HBB, SEQ ID No. 10, or the genes coding for non-human hemoglobins may be located on a yeast plasmid (such as pSP-GM1). The hemoglobin genes may be overexpressed.

The method of overexpression can be achieved by: introducing 1, 2, 3, 4 or more copies of genes encoding hemoglobin into the yeast genome or by multi-copy of the plasmid; or by substituting a native promoter of the hemoglobin gene by a strong constitutive promoter (such as promoters of genes TEF1 or PGK1); or by modifying the native promoter of the hemoglobin gene to increase the transcription of the hemoglobin gene or by modifying the native terminator of the hemoglobin gene to increase the half-life of the hemoglobin gene transcript; or by modifying the proteins involved in regulation of hemoglobin gene translation (either repressors or activators) to result in increased level of hemoglobin.

In one example, one copy of the human HBA gene may be cloned under the strong promoter PGK1, a second copy of the human HBA gene may be cloned under the strong promoter TEF1, and the human HBB gene may be cloned under the strong promoter PGK1.

Non-human hemoglobins containing heme as a cofactor and a globin part that reversibly binds gaseous ligands may for example be bovine hemoglobin or hemoglobins from other vertebrates, plant hemoglobins (for example, from soy, pea, rice or barley, etc). With gaseous ligands is here meant oxygen, carbon dioxide, carbon monoxide, and nitric oxide. As non-human heme proteins have similar properties to human hemoglobin, such as that they carry heme as a cofactor, they require heme for their activity, and are therefore possible to produce in the above-described modified yeast cell. Plant hemoglobins (non-symbiotic plant hemoglobins from, for example soy, pea, rice or barley) can be expressed in a yeast strain as described herein.

A genetically modified yeast cell comprising a genetic modification comprising overexpression of human hemoglobin genes, overexpression of the HEM3 gene, overexpression of the AHSP gene, deletion of the ROX1 gene, deletion of the HMX1 gene, deletion of the VPS10 gene, and deletion of the PEP4 gene, show a yield of intracellular human hemoglobin relative to the total yeast protein produced during glucose fermentation being as high as 18%. This is so far, what is known, the highest production of human hemoglobin reported in yeast using glucose as a substrate. This hemoglobin production was accompanied with increased oxygen consumption rates and higher glycerol yield, which is hypothesized to be the response of the yeast cell to balance NADH levels under high protein production conditions or oxygen limitation.

Such a genetically modified yeast cell may be used to provide polypeptides that have hemoglobin α activity, being at least 95% identical to SEQ ID No. 11, and polypeptides that have hemoglobin β activity, being at least 95% identical to SEQ ID No. 12.

In one alternative embodiment, the genetically modified yeast cell may comprise genes coding for hemoglobin-based oxygen carriers (HBOCs), myoglobin or P450 enzymes.

Myoglobin is a heme-containing protein encoded by the MB gene. Human P450 2S1 is a heme-containing protein encoded by the CYP2S1 gene. The genes coding for HBOCs, myoglobin or P450 enzymes may be located on a yeast plasmid (such as pESC-URA or pSP-GM1).

According to a second aspect there is provided a genetically modified yeast cell as described above, wherein the yeast cell is selected from a group comprising Saccharomyces cerevisiae, Pichia pastoris, Hansenula polymorpha and Yarrowia lipolytica.

In a preferred embodiment the yeast cell is Saccharomyces cerevisiae.

Such a modified Saccharomyces cerevisiae yeast cell may be: Δrox1Δvps10Δhmx1Δpep4/HEM3+AHSP/ααβ, carrying the above discussed genetic modifications, i.e. deletion of genes ROX1, VPS10, HMX1, and PEP4 and overexpression of the HEM3 gene, the AHSP gene and the human hemoglobin genes ((two copies of HBA (encoding hemoglobin subunit alpha, a) and one copy of HBB (encoding hemoglobin subunit beta, β)), and may be used for intracellular human hemoglobin production.

According to a third aspect there is provided a product produced by using the genetically modified yeast cell describe above.

Such a product may be a human or non-human hemoglobin produced by the modified yeast cell described above having human hemoglobin genes in its genome, the hemoglobins being secreted into a medium. Alternatively, the product may be hemoglobin-based oxygen carriers (HBOCs), myoglobin or P450 enzymes produced by the modified yeast cell described above having genes coding for such proteins in its genome. The product may for example be a blood substitute or a food additive or substitute.

### BRIEF DESCRIPTION OF DRAWINGS

The invention will be described in greater detail in the following, with reference to the embodiments that are shown in the attached drawings, in which:

FIG. 1. Shows the schematic overview of construction of yeast strains (i.e. genetically modified yeast cells in accordance with the present disclosure) with reduced degradation of heme and hemoglobin.

FIG. 2. Shows total porphyrins of constructed yeast strains. Strains: 1—WT/empty vectors, control; 2—WT/HEM3/ααβ; 3—Δrox1/HEM3/ααβ, 4—Δrox1Δvps10/HEM3/ααβ, 5—Δrox1Δvps10Δhmx1/HEM3/ααβ, 6—Δrox1Δvps10Δhmx1Δpep4/HEM3/ααβ, 7—Δrox1Δvps10Δhmx1Δpep4/HEM3+AHSP/ααβ.

FIG. 3. Shows hemoglobin expression in constructed strains studied by Western blotting. A) Protein SDS PAGE gel of TCA protein extracts of yeast strains expressing human hemoglobin HbA. B) Western blotting using anti-alpha hemoglobin antibodies. Strains: 1—WT/empty vectors, control; 2—WT/HEM3/ααβ; 3—Δrox1Δvps10Δhmx1Δpep4/HEM3/ααβ, 4—Δrox1Δvps10Δhmx1Δpep4/HEM3+AHSP/ααβ.

FIG. 4. Shows ROS production by constructed strains at 6 hours of fermentation. Strains: 1—WT/empty vectors, control; 2—WT/HEM3/ααβ; 3—Δrox1/HEM3/ααβ, 4—Δrox1Δvps10/HEM3/ααβ, 5—Δrox1Δvps10Δhmx1/HEM3/ααβ, 6—Δrox1Δvps10Δhmx1Δpep4/HEM3/ααβ, 7—Δrox1Δvps10Δhmx1Δpep4/HEM3+AHSP/ααβ.

FIG. 5. Shows ROS accumulation by constructed strains at 24 hours of fermentation. Samples were collected at 24 hours of glucose fermentation, and ROS were detected by dihydrorhodamine 123 staining. 5000 cells of each strain were analyzed by flow cytometry. Strains: 1—WT/empty vectors, control; 2—WT/HEM3/ααβ; 3—Δrox1/HEM3/ααβ, 4—Δrox1Δvps10/HEM3/ααβ, 5—Δrox1Δvps10Δhmx1/HEM3/ααβ, 6—Δrox1Δvps10Δhmx1Δpep4/HEM3/ααβ, 7—Δrox1Δvps10Δhmx1Δpep4/HEM3+AHSP/ααβ.

FIG. 6. Shows growth rate of constructed strains. Strains: 1—WT/empty vectors, control; 2—WT/HEM3/ααβ; 3—Δrox1/HEM3/ααβ, 4—Δrox1Δvps10/HEM3/ααβ, 5—Δrox1Δvps10Δhmx1/HEM3/ααβ, 6—Δrox1Δvps10Δhmx1Δpep4/HEM3/ααβ, 7—Δrox1Δvps10Δhmx1Δpep4/HEM3+AHSP/ααβ.

FIG. 7. Shows Western blotting of TCA protein extracts using anti-hemoglobin α antibodies. Strains: 1—WT/empty vectors, control; 2—WT/HEM3/ααβ; 3—Δrox1/HEM3/ααβ, 4—Δrox1Δvps10Δhmx1/HEM3/ααβ, 5—Δrox1Δvps10Δhmx1Δpep4/HEM3/ααβ, 6—Δrox1Δvps10Δhmx1Δpep4/HEM3+AHSP/ααβ.

FIG. 8. Shows hemoglobin expressed in yeast binds carbon monoxide. Absorption spectra of supernatant of yeast crude extracts treated with CO-releasing compound CORM-3. Strains: 1—WT/HEM3/ααβ; 2—Δrox1Δvps10Δhmx1Δpep4/HEM3+AHSP/ααβ.

FIG. 9. Shows bilirubin biosensor expressed in yeast.
A) The mCherry-UnaG construct was expressed in yeast under promoter PGK1 to study the accumulation of heme/hemoglobin degradation product bilirubin.
B) mCherry-UnaG green fluorescence yield in different strains expressing HbA: B1) and B2) wild-type (WT); B3) Δrox1; B4) Δrox1Δvps10; B5) Δrox1Δvps10Δhmx1; B6) Δrοx1Δvps10Δpep4. Two biological replicates were used in analysis.

FIGS. 10A-B. Shows Hbfusion expression results in lower bilirubin formation compared to HbA on FIG. 9B3. mCherry-UnaG green fluorescence yield in Δrox1: 10A) without Hbfusion; 10B) with Hbfusion. Two biological replicates were used in analysis.

FIG. 11. Shows AHSP strain had increased cell size. Cell volume estimated by CASY Model TT Cell Strains: 1—WT/HEM3/ααβ; 2—Δrox1Δvps10Δhmx1Δpep4/HEM3+AHSP/ααβ.

FIG. 12. Shows cell size of hemoglobin production strains. Cell volume of different yeast strains estimated by CASY Model TT Cell Counter at different time of glucose fermentation.

FIGS. 13A-G. Shows expression of GFP-Hbfusion reporter construct in yeast. GFP-fluorescence yield per biomass of strains carrying GFP-Hbfusion reporter during glucose fermentation was analyzed in 2 biological replicates in BioLector®.

FIG. 14. Shows increased iron supplementation in the medium improves hemoglobin production and reduces hemoglobin degradation.
A1, A2) Porphyrin synthesis in SD medium with 100 and 200 μM Fe³⁺.
B1, B2, B3, B4) Protein SDS gel and Western blotting of TCA extracts of WT and AHSP strains using anti-Hb alpha and anti-Gapdh antibodies.
C1, C2) Bilirubin biosensor fluorescence is lower in the medium with higher amount of Fe³⁺

FIG. 15. Shows growth, glucose consumption, and metabolites yield of 15A: WT/HEM3/ααβ and 15B: Δrox1Δvps10Δhmx1Δpep4/HEM3+AHSP/ααβ strains.

FIG. 16. Shows expression of hemoglobin and total protein content of hemoglobin strains.
A1, A2) Estimation of hemoglobin production by Western blotting using anti-hemoglobin α antibodies.
B) Relative protein content of cells during bioreactors cultivation at 24 and 48 hours.

FIG. 17. Shows hemoglobin production in different strains at 27 and 48 hours during the fermentation in bioreactors.
A) Western blotting using anti-hemoglobin α antibodies. Strains: 1, 2—WT/HEM3/ααβ; 3, 4—Δrox1Δvps10Δhmx1Δpep4/HEM3+AHSP/ααβ; 5, 6-Δrox1Δvps10Δhmx1Δpep4/HEM3/ααβ.
B) Hb content relative to total protein content.

FIG. 18. Shows the expression of different heme proteins in WT and Δrox1Δvps10Δhmx1Δpep4 strains determined by Western blotting. In both strains, the heme-protein genes were co-expressed with the HEM3 gene of S. cerevisiae. A) MYG-BOV: expression of bovine myoglobin encoded by the MB (myoglobin) gene of Bos taurus; B) HBL-HOR: expression of non-symbiotic hemoglobin encoded by GLB1 gene of Hordeum vulgare; C) CYP2S1-Hs: expression of cytochrome P450 2S1 encoded by CYP2S1 of Homo sapiens. The Gapdh (Glyceraldehyde dehydrogenase) signal was used to normalize the data.

FIG. 19. Shows expression of hemoglobin fusion via secretory pathway in three different yeast strains.
A) Hemoglobin was expressed as a fusion peptide (ay-globin fusion with S. cerevisiae native α-factor leader sequence to direct it to secretory pathway) in CENPK113-11c rox1 vps10 hmx1 pep4, 184M, and INVSc1 strains.
B) SDS protein gel and Western blotting results using polyclonal antibodies against γ-globin chain.

### DETAILED DESCRIPTION

Embodiments of the invention with further developments described in the following are to be regarded only as examples and are in no way intended to limit the scope of the protection provided by the patent claims. (Note that the wording “this study” herein refers to the work disclosed in this patent application.)

### Experimental

### The Deletion of the Transcriptional Repressor ROX1 Increases Intracellular Hemoglobin Levels

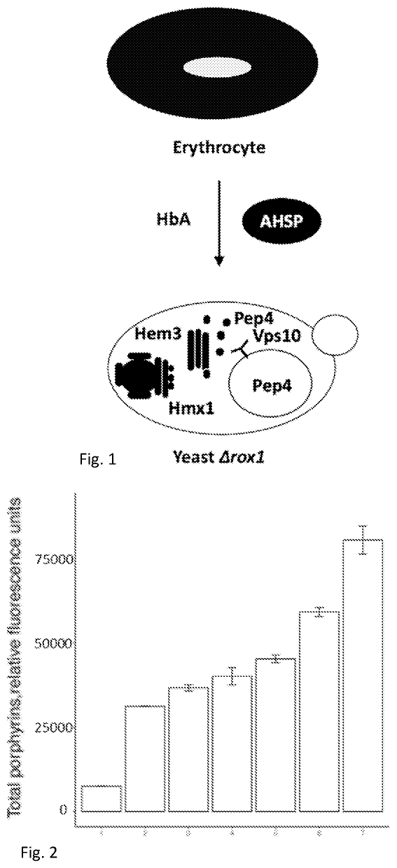

The transcriptional factor Rox1, which is an activator of hypoxic genes, is also a repressor of heme biosynthesis pathway. The Rox1 inhibits the transcription of the HEM13 gene. Previous studies found that the deletion of ROX1 gene or the changes of its expression in S. cerevisiae results in improved production of heterologous proteins, such as α-amylase and insulin precursor (Liu L, Zhang Y, Liu Z et al. Improving heterologous protein secretion at aerobic conditions by activating hypoxia-induced genes in Saccharomyces cerevisiae. FEMS Yeast Res. 2015. 15 (7). pii: fov070; and Huang M, Bao J, Hallstrom B M, et al. Efficient protein production by yeast requires global tuning of metabolism. Nat Commun. 2017. 8 (1): 1131.). The deletion of the ROX1 gene also resulted in increased intracellular levels of heme (Zhang T, Bu P, Zeng J et al. Increased heme synthesis in yeast induces a metabolic switch from fermentation to respiration even under conditions of glucose repression. J Biol Chem. 2017. 292 (41): 16942-16954.). When transformed with the plasmids for recombinant Hemoglobin A (HbA) expression (plYC04+HEM3 and pSP-GM1+ααβ, see FIG. 1 and Table 1 below), which carry HEM3 gene human genes encoding hemoglobin subunit alpha (two copies of HBA, α) and subunit beta (one copy of HBB, β), Δrox1 strain displayed darker red pigmentation, a consequence of higher porphyrins accumulation compared to the WT (wild type) carrying hemoglobin expression plasmids on the medium with heme precursor 5-aminolevulinic acid (5-ALA). The Δrox1 strain was found to accumulate 1.2 times higher amounts of total porphyrins compared to WT carrying hemoglobin expression plasmids (FIG. 2). Due to its superior characteristics in porphyrins production, the Δrox1 strain was selected as the background strain for further engineering strategies. Yeast Strains used in this study, see Table 2 below.

TABLE 1
Oligonucleotides and plasmids used in this study used to construct
plasmids and strains of present invention.

| | Oligonucleotide | |
|---|---|---|
| Oligonucleotide name | sequence, 5′-3′ | Reference |
| Del-HMX1-1 | CATACTCTCTTGCTTAGTC | This study |
| | TAAGGAGGAGCTATTTAA | |
| | CAGTGCACGACAACCCTT | |
| | AATTACCGTTC (SEQ ID | |
| | NO: 15) | |
| Del1-rev | CAACCTATTAATTTCCCCT | This study |
| | CGTCAAAAATAAGGTTAT | |
| | CAAGTGAGAAATCACCAT | |
| | GAG (SEQ ID NO: 16) | |
| Del2-for | GGCAAAACAGCATTCCAG | This study |
| | GTATTAGAAGAATATCCT | |
| | GATTCAGGTGAAAATATT | |
| | G (SEQ ID NO: 17) | |
| Del2-HMX1-2 | AGCTATCTCAGGGTAGAT | This study |
| | AATAGGGCTGTAAAAACC | |
| | TCTCACATGGGATCTGAT | |
| | ATACCGTTCGTATAGCATA | |
| | C (SEQ ID NO: 18) | |
| VPS10-1 | ATTACTTCATTTTGTCTATT | This study |
| | CTCTTTGGGCCTTACTTCT | |
| | CATTCCGACAACCCTTAAT | |
| | TACCGTTC (SEQ ID NO: | |
| | 19) | |
| VPS10-2 | TATCTACTCTATGTAAAGT | This study |
| | AATCTCTCTACTGGTTTTC | |
| | GTTAGATGGGATCTGATA | |
| | TACCGTTCGTATAGCATAC | |
| | (SEQ ID NO: 20) | |
| PEP4-4 | ATGTTCAGCTTGAAAGCA | This study |
| | TTATTGCCATTGGCCTTGT | |
| | TGTTGGTCAGGACAACCC | |
| | TTAATTACCGTTC (SEQ ID | |
| | NO: 21) | |
| PEP4-2 | TATAAAAGCTCTCTAGATG | This study |
| | GCAGAAAAGGATAGGGC | |
| | GGAGAAGTAAGGGATCT | |
| | GATATACCGTTCGTATAG | |
| | CATAC (SEQ ID NO: 22) | |
| AHSP-1 | CCTGGATCCATGGCTTTAT | This study |
| | TAAAAGCCAATAAGGAT | |
| | (SEQ ID NO: 23) | |
| AHSP-2 | TTTCTCGAGCTAAGAAGA | This study |
| | TGGTGGTGGATGAGATG | |
| | (SEQ ID NO: 24) | |
| HbF-GFP-1 | CTACTTTTTACAACAAATA | This study |
| | TAAAACAAGGATCCATGC | |
| | GAATCCCCGGGTTAATTA | |
| | AC (SEQ ID NO: 25) | |
| HbF-GFP-2 | GTTTTATCGGCAGGAGAT | This study |
| | AAGACTTTGTATAGTTCAT | |
| | CCATGCCATG (SEQ ID | |
| | NO: 26) | |
| HbF-GFP-3 | CATGGCATGGATGAACTA | This study |
| | TACAAAGTCTTATCTCCTG | |
| | CCGATAAAAC (SEQ ID | |
| | NO: 27) | |
| H3AFHb-2 | GCGGTACCAAGCTTACTC | This study |
| | GAGTCAATGATATCTGGA | |
| | AC (SEQ ID NO: 28) | |
| FDD-B | AAAGGATCCATGCGGTGA | This study |
| | GCAAGGGCGAG (SEQ ID | |
| | NO: 29) | |
| FDD-X | AAACTCGAGTCATTCCGTC | This study |
| | GCCCCCCGGTAG (SEQ ID | |
| | NO: 30) | |
| Alpha-1 | CACATACATAAACTAAAA | This study |
| | GGTACCAACAAAATGAGA | |
| | TTTCCATC (SEQ ID NO: | |
| | 31) | |
| Alpha-2 | TCGGCAGGAGATAAGACT | This study |
| | TTTGGTTCACCTTCTTC | |
| | (SEQ ID NO: 32) | |
| Fusion-1 | GAAGAAGGTGAACCAAA | This study |
| | AGTCTTATCTCCTGCCGA | |
| | (SEQ ID NO: 33) | |
| Fusion-2 | GTTTCTAGACTCGAGGCT | This study |
| | AGCTCAATGATATCTGGA | |
| | AC (SEQ ID NO: 34) | |
| HEM3CPOT-1 | AGCAACCGTTGGCATGGA | This study |
| | TCCCTAGGAATTGGAGCG | |
| | AC (SEQ ID NO: 35) | |
| HEM3CPOT-2 | GATCTGGCCGGCCGGATC | This study |
| | CCCGCACACACCATAGCTT | |
| | C (SEQ ID NO: 36) | |
| H3AFHb-1 | CAACAAATATAAAACAAG | This study |
| | GATCCAACAAAATGAGAT | |
| | TTCCATC (SEQ ID NO: 37) | |
| H3AFHb-2 | GCGGTACCAAGCTTACTC | This study |
| | GAGTCAATGATATCTGGA | |
| | AC (SEQ ID NO: 38) | |
| Plasmid name | | Reference |
| pDel1 | | Wenning et al., 2017 |
| pDel2 | | Wenning et al., 2017 |
| plYC04 | | Chen et al., 2013 |
| plYC04 + HEM3 | | Liu et al., 2014 |
| (plasmid H3) | | |
| pSP-GM1 | | Chen et al., 2012 |
| pSP-GM1 + ααβ | | Liu et al., 2014 |
| (plasmid B/A/A) | | |
| plYC04 + HEM3 + | | This study |
| AHSP | | |
| p416TEFGFP | | Jensens et al., 2017 |
| plYC04 + HEM3 + | | This study |
| GFP-Hbfusion | | |
| mCherry-FDD | | Addgene #80629, |
| | | Navarro et al., 2016 |
| plYC04 + HEM3 + | | This study |
| mCherry-UnaG | | |
| plYC04 + HEM3 + α- | | This study |
| leader-Hbfusion | | |
| CPOTud | | Liu et al., 2012 |
| CPOT + α-leader- | | This study |
| Hbfusion + HEM3 | | |
| pESC-URA + CYP2S1 | | This study |
| pESC-URA + MYG-BOV | | This study |
| pESC-URA + HBL-HOR | | This study |

TABLE 2
Yeast strains used in this study.

| Strain name | Description | Reference |
|---|---|---|
| CEN.PK 113-11C (WT) | MATa his3Δ1 ura 3-52 | Entian and Kotter, 1998 |
| | MAL2-8c SUC2 | |
| WT/plYC04/pSP-GM1 | Control strain carrying | This study |
| | empty vectors | |
| WT/H3/ααβ | WT strain transformant | Liu et al., 2014 |
| | with plasmids | |
| | plYC04 + HEM3 and pSP- | |
| | GM1 + ααβ (plasmid | |
| | B/A/A) | |
| CEN.PK 113-11C Δrox1 | MATa his3Δ1 ura 3-52 | Liu et al., 2015 |
| (Δrox1) | MAL2-8c SUC2 Δrox1 | |
| Δrox1/H3/ααβ | Δrox1 strain transformant | This study |
| | with plasmids | |
| | plYC04 + HEM3 and pSP- | |
| | GM1 + ααβ (plasmid | |
| | B/A/A) | |
| Δrox1 Δvps10 | MATa his3Δ1 ura 3-52 | This study |
| | MAL2-8c SUC2 Δrox1 | |
| | Δvps10 | |
| Δrox1Δvps10/H3/ααβ | Δrox1Δvps10 strain | This study |
| | transformant with | |
| | plasmids plYC04 + HEM3 | |
| | and pSP-GM1 + ααβ | |
| | (plasmid B/A/A) | |
| Δrox1Δvps10Δhmx1 | MATa his3Δ1 ura 3-52 | This study |
| | MAL2-8c SUC2 Δrox1 | |
| | Δvps10 Δhmx1 | |
| Δrox1Δvps10Δhmx1/H3/ααβ | Δrox1Avps10Δhmx1 strain | This study |
| | transformant with | |
| | plasmids plYC04 + HEM3 | |
| | and pSP-GM1 + ααβ | |
| | (plasmid B/A/A) | |
| Δrox1Δvps10Δpep4 | MATa his3Δ1 ura 3-52 | This study |
| | MAL2-8c SUC2 Δrox1 | |
| | Δvps10 Δpep4 | |
| Δrox1Δvps10Δhmx1Δpep4 | MATa his3Δ1 ura 3-52 | This study |
| | MAL2-8c SUC2 Δrox1 | |
| | Δvps10 Δhmx1 Δpep4 | |
| Δrox1Δvps10Δhmx1Δpep4/ | Δrox1Δvps10Δhmx1Δpep4 | This study |
| H3/ααβ | strain transformant with | |
| | plasmids plYC04 + HEM3 | |
| | and pSP-GM1 + ααβ | |
| | (plasmid B/A/A) | |
| Δrox1Δvps10Δhmx1Δpep4/ | Δrox1Δvps10Δhmx1Δpep4 | This study |
| H3 + AHSP/ααβ, AHSP | strain transformant with | |
| | plasmids | |
| | plYC04 + HEM3 + AHSP and | |
| | pSP-GM1 + ααβ (plasmid | |
| | B/A/A) | |
| WT/H3 + mCherry-UnaG/ | WT transformant with | This study |
| pSP-GM1 | plasmids | |
| | plYC04 + HEM3 + mCherry- | |
| | UnaG and pSP-GM1 | |
| WT/H3 + mCherry-UnaG/ | WT transformant with | This study |
| ααβ | plasmids | |
| | plYC04 + HEM3 + mCherry- | |
| | UnaG and pSP-GM1 + ααβ | |
| | (plasmid B/A/A) | |
| Δrox1/H3 + mCherry-UnaG/ | Δrox1 transformant with | This study |
| ααβ | plasmids | |
| | plYC04 + HEM3 + mCherry- | |
| | UnaG and pSP-GM1 + ααβ | |
| | (plasmid B/A/A) | |
| Δrox1Δvps10/H3 + mCherry- | Δrox1Δvps10 | This study |
| UnaG/ααβ | transformant with | |
| | plasmids | |
| | plYC04 + HEM3 + mCherry- | |
| | UnaG and pSP-GM1 + ααβ | |
| | (plasmid B/A/A) | |
| Δrox1Δvps10Δhmx1/H3 + | Δrox1Δvps10Δhmx1 | This study |
| mCherry-UnaG/ααβ | transformant with | |
| | plasmids | |
| | plYC04 + HEM3 + mCherry- | |
| | UnaG and pSP-GM1 + ααβ | |
| | (plasmid B/A/A) | |
| Δrox1Δvps10Δpep4/H3 + | Δrox1Δvps10Δpep4 | This study |
| mCherry-UnaG/ααβ | transformant with | |
| | plasmids | |
| | plYC04 + HEM3 + mCherry- | |
| | UnaG and pSP-GM1 + ααβ | |
| | (plasmid B/A/A) | |
| WT/H3 + GFP-Hbfusion/ | WT transformant with | This study |
| pSP-GM1 | plasmids | |
| | plYC04 + HEM3 + GFP- | |
| | Hbfusion and pSP-GM1 | |
| Δrox1/H3 + GFP-Hbfusion/ | Δrox1 transformant with | This study |
| pSP-GM1 | plasmids | |
| | plYC04 + HEM3 + GFP- | |
| | Hbfusion and pSP-GM1 | |
| Δrox1Δvps10/H3 +GFP- | Δrox1Δvps10 | This study |
| Hbfusion/pSP-GM1 | transformant with | |
| | plasmids | |
| | plYC04 + HEM3 + GFP- | |
| | Hbfusion and pSP-GM1 | |
| Δrox1Δvps10Δpep4/H3 + | Δrox1Δvps10Δpep4 | This study |
| GFP-Hbfusion/pSP-GM1 | transformant with | |
| | plasmids | |
| | plYC04 + HEM3 + GFP- | |
| | Hbfusion and pSP-GM1 | |
| Δrox1Δvps10Δhmx1Δpep4/ | Δrox1Δvps10Δhmx1Δpep4 | This study |
| H3 + GFP-Hbfusion/pSP-GM1 | transformant with | |
| | plasmids | |
| | plYC04 + HEM3 + GFP- | |
| | Hbfusion and pSP-GM1 | |
| INVSc1 | MATa his3D1 leu2 trp1- | Invitrogen ™ |
| | 289 ura3-52 MAT his3D1 | |
| | leu2 trp1-289 ura3-52 | |
| 184M | Improved by UV | Huang et al., 2015 |
| | mutagenesis secretion | |
| | strain | |
| Δrox1Δvps10Δhmx1Δpep4/ | Δrox1Δvps10Δhmx1Δpep4 | This study |
| plYC04 + HEM3 + α-leader- | transformant carrying the | |
| Hbfusion/pSP-GM1 | expression plasmid for | |
| | secreted Hbfusion | |
| | (plYC04 + HEM3 + α-leader- | |
| | Hbfusion) and pSP-GM1 | |
| 184M/CPOT + α-leader- | 184M transformant | This study |
| Hbfusion + HEM3 | carrying the expression | |
| | plasmid for secreted | |
| | Hbfusion (CPOT + α- | |
| | leader-Hbfusion + HEM3) | |
| INVSc1/plYC04 + HEM3 + α- | INVSc1 transformant | This study |
| leader-Hbfusion/pSP-GM1 | carrying the expression | |
| | plasmid for secreted | |
| | Hbfusion | |
| | (plYC04 + HEM3 + α-leader- | |
| | Hbfusion) and pSP-GM1 | |
| Δrox1Δvps10Δhmx1Δpep4/ | Δrox1Δvps10Δhmx1Δpep4 | This study |
| pESC-URA + CYP2S1/ | transformant carrying the | |
| plYC04 + HEM3 | expression plasmids for | |
| | P450 (pESC-URA + CYP2S1) | |
| | and HEM3 | |
| | (plYC04 + HEM3) | |
| WT/pESC-URA + CYP2S1/ | WT transformant carrying | This study |
| plYC04 + HEM3 | the expression plasmids | |
| | for P450 (pESC- | |
| | URA + CYP2S1) and HEM3 | |
| | (plYC04 + HEM3) | |
| Δrox1Δvps10Δhmx1Δpep4/ | Δrox1Δvps10Δhmx1Δpep4 | This study |
| pESC-URA + MYG-BOV/ | transformant carrying the | |
| plYC04 + HEM3 | expression plasmids for | |
| | bovine myoglobin (pESC- | |
| | URA + MYG-BOV) | |
| | and HEM3 | |
| | (plYC04 + HEM3) | |
| WT/pESC-URA + MYG-BOV/ | WT transformant carrying | This study |
| plYC04 + HEM3 | the expression plasmids | |
| | for bovine myoglobin | |
| | (pESC-URA + MYG-BOV) | |
| | and HEM3 | |
| | (plYC04 + HEM3) | |
| Δrox1Δvps10Δhmx1Δpep4/ | Δrox1Δvps10Δhmx1Δpep4 | This study |
| pESC-URA + HBL-HOR/ | transformant carrying the | |
| plYC04 + HEM3 | expression plasmids for | |
| | plant hemoglobin (pESC- | |
| | URA + HBL-HOR) | |
| | and HEM3 | |
| | (plYC04 + HEM3) | |
| WT/pESC-URA + HBL-HOR/ | WT transformant carrying | This study |
| plYC04 + HEM3 | the expression plasmids | |
| | for plant hemogobin | |
| | (pESC-URA + HBL-HOR) and | |
| | HEM3 (plYC04 + HEM3) | |

The deletion of genes involved in heme and hemoglobin degradation, alone and in combination with overexpression of AHSP, results in increased Hb levels

In order to improve the production of hemoglobin we followed a strategy for the reducing both hemoglobin and heme intracellular degradation (FIG. 1). We deleted the VPS10 (encoding type I transmembrane sorting receptor for multiple vacuolar hydrolases), PEP4 (encoding vacuolar aspartyl protease [proteinase A]), and HMX1 (encoding ER localized heme oxygenase) genes in S. cerevisiae genome and overexpressed the human α-hemoglobin-stabilizing protein, encoded by the AHSP gene (FIG. 1). The three deletions were introduced by the Cre-lox system and kanMX as selectable marker (Δvps10, Δhmx1, and Δpep4) into the Δrox1 strain background. After each deletion, the kanMX marker was removed by the induction of Cre-recombinase expression (as described in section “Materials and methods” herein). The VPS10 and PEP4 genes are known to affect the targeting of misfolded proteins to the vacuole and their vacuolar degradation, and while HMX1 gene is a part of heme degradation pathway (Hong E, Davidson A R, Kaiser C A. A pathway for targeting soluble misfolded proteins to the yeast vacuole. J Cell Biol. 1996. 135 (3): 623-633; Protchenko O, Philpott C C. Regulation of intracellular heme levels by HMX1, a homologue of heme oxygenase, in Saccharomyces cerevisiae. J Biol Chem. 2003. 278 (38): 36582-7; and Marques M, Mojzita D, Amorim M A et al. The Pep4p vacuolar proteinase contributes to the turnover of oxidized proteins but PEP4 overexpression is not sufficient to increase chronological lifespan in Saccharomyces cerevisiae. Microbiology. 2006. 152 (Pt 12): 3595-3605.). The Pep4p vacuolar proteinase contributes to the turnover of oxidized proteins.

The AHSP gene was cloned and expressed on plYC04+HEM3 plasmid (Table 1) as a synthetic peptide with its codons optimized for its expression in S. cerevisiae host. All deletion strains were transformed with adult hemoglobin (HbA) expression plasmids (plYC04+HEM3 and pSP-GM1+ααβ or plYC04+HEM3+AHSP and pSP-GM1+ααβ [Table 1]).

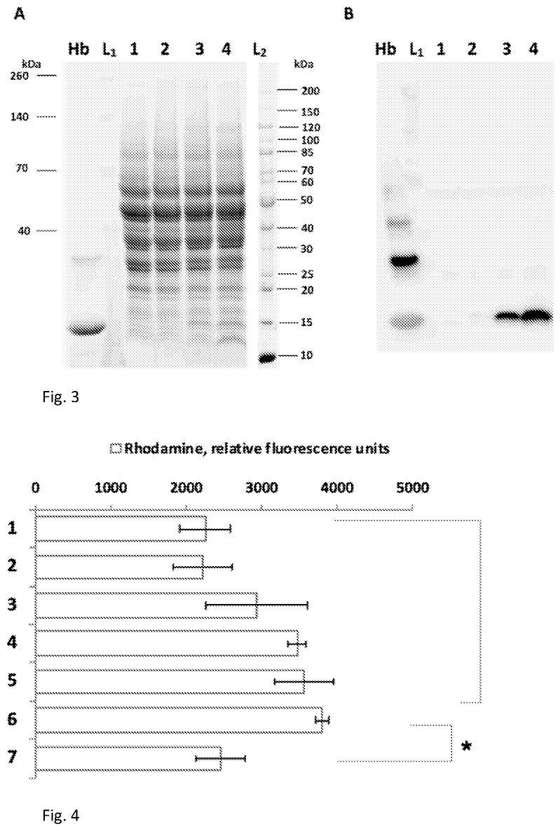

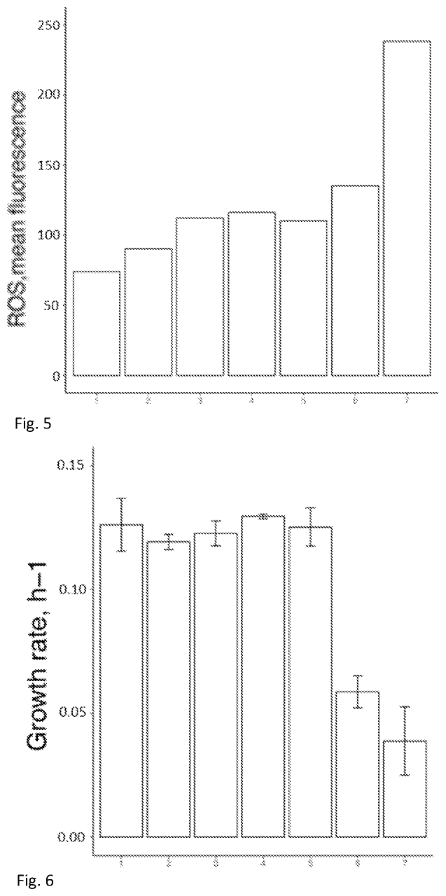

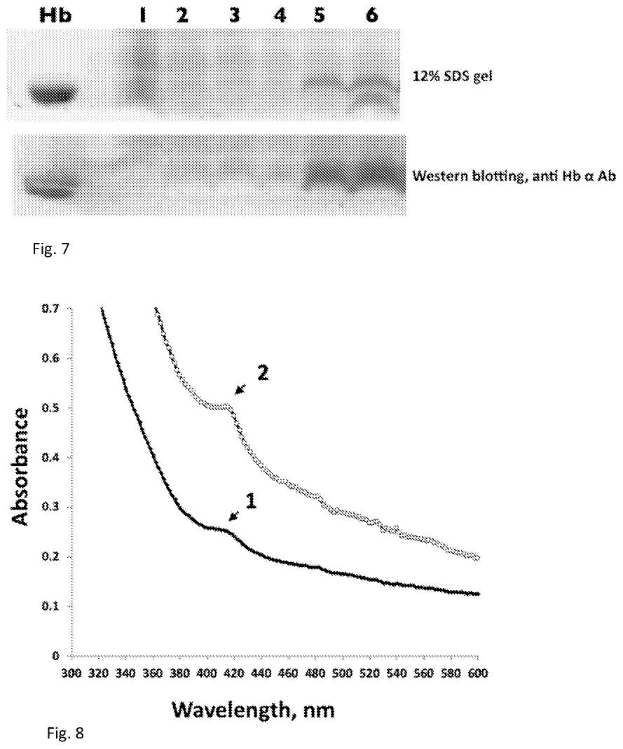

The production of both porphyrins and hemoglobin was increased stepwise and reached the highest level in the AHSP overexpression strain (FIG. 2). In erythrocytes and in E. coli, AHSP prevents α-globin from degradation by forming a stable complex with free α-globin subunit (Kihm A J, Kong Y, Hong W et al. An abundant erythroid protein that stabilizes free alpha-haemoglobin. Nature. 2002. 417 (6890): 758-763; Vasseur-Godbillon C, Hamdane D, Marden M C et al. High-yield expression in Escherichia coli of soluble human alpha-hemoglobin complexed with its molecular chaperone. Protein Eng Des Sel. 2006. 19 (3): 91-97.). In our yeast model, the overexpression of AHSP gene increased the hemoglobin production by 58% (FIG. 3). The total porphyrins level at 24 hours in the Δrox1Δvps10Δhmx1Δpep4/HEM3+AHSP/ααβ strain was 2.6 times higher compared to the WT/HEM3/ααβ strain (FIG. 2). While the Δrox1Δvps10Δhmx1Δpep4/HEM3+AHSP/ααβ strain displayed slightly reduced level of ROS at early stage of glucose fermentation, at 6 hours (FIG. 4), which could be due to the antioxidant AHSP activity (Yu X, Kong Y, Dore L C et al. An erythroid chaperone that facilitates folding of alpha-globin subunits for hemoglobin synthesis. J Clin Invest. 2007. 117 (7): 1856-1865; and Kiger L, Vasseur C, Domingues-Hamdi E et al. Dynamics of α-Hb chain binding to its chaperone AHSP depends on heme coordination and redox state. Biochim Biophys Acta. 2014.), at 24 hours we observed that the increased hemoglobin production positively correlated with increased ROS production (FIG. 2, FIG. 5), as it occurs in other protein production yeast (Shimizu and Hendershot. Oxidative Folding: Cellular Strategies for Dealing With the Resultant Equimolar Production of Reactive Oxygen Species. Antioxid Redox Signal. 2009. 11 (9): 2317-31; Tyo K E, Liu Z, Petranovic D, Nielsen J. Imbalance of heterologous protein folding and disulfide bond formation rates yields runaway oxidative stress. BMC Biol. 2012. 10:16.). The increased hemoglobin production was also accompanied with reduced growth rate of yeast (FIG. 6) probably due to higher protein production in the cell or/and toxicity of hemoglobin to yeast. After the introduction of pep4 deletion, the α-globin band of hemoglobin was readily detectable by both protein SDS gel and Western blotting (FIG. 3, FIG. 7).

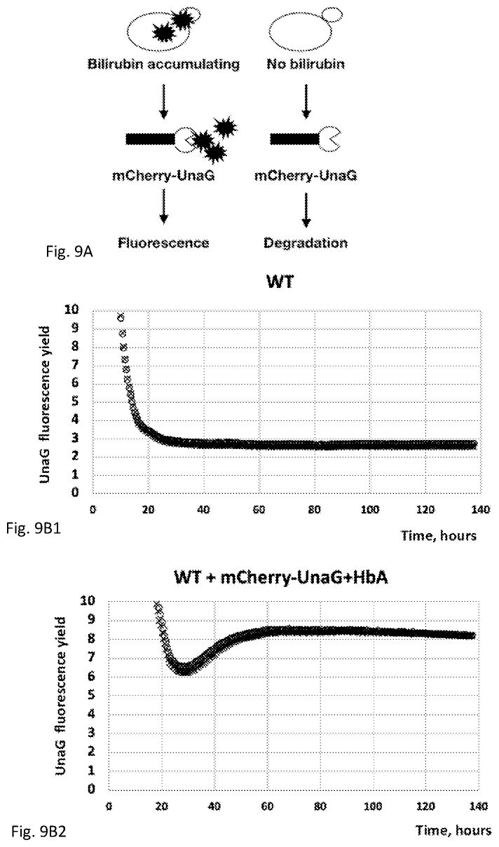

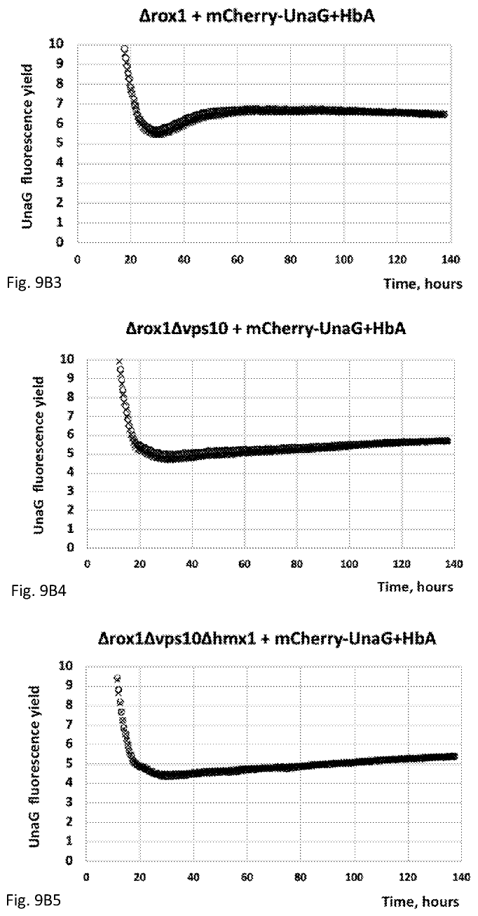

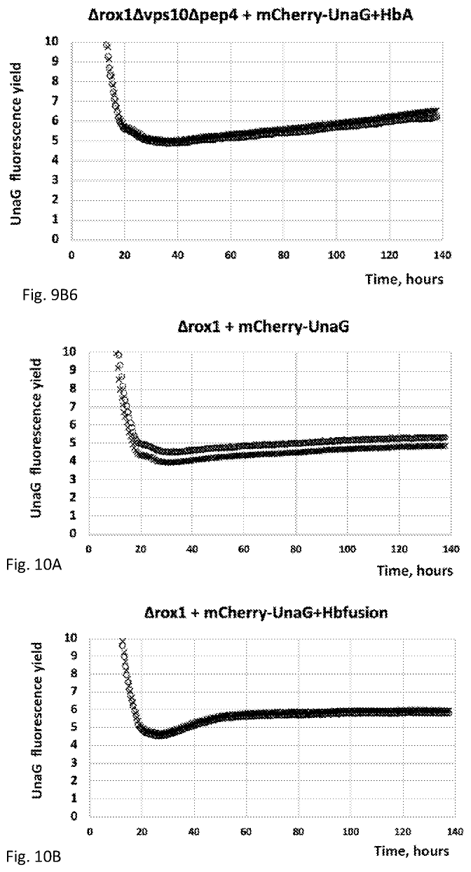

The hemoglobin activity in yeast was detected by the absorption spectra of carboxyhemoglobin (Hb-CO) formed after the treatment of cellular extracts with carbon monoxide-generating compound (CORM-3) (FIG. 8). The cell free extract of Δrox1 Δvps10Δhmx1Δpep4/HEM3+AHSP/ααβ strain treated with CO-generating compound (CORM-3) had 3-times higher absorption peak at 419 nm (corresponding to the Hb-CO) compared to the WT/HEM3/ααβ strain (FIG. 8).

### Lowered Amount of Hemoglobin-Degradation Product Bilirubin is Formed in the Engineered Strains

The previously described engineered strains were analyzed for the accumulation of hemoglobin degradation product, bilirubin. This was done by using bilirubin-binding biosensor UnaG protein from eel muscle developed previously (Kumagai A, Ando R, Miyatake H et al. A bilirubin-inducible fluorescent protein from eel muscle. Cell. 2013. 153 (7): 1602-11.). The mCherry-UnaG fusion from the plasmid mCherry-FDD, that was developed for red and green fluorescence assays of bilirubin (Navarro R, Chen L C, Rakhit R et al. A Novel Destabilizing Domain Based on a Small-Molecule Dependent Fluorophore. ACS Chem Biol. 2016. 11 (8): 2101-4.), was cloned under yeast promoter PGK1 on the vector plYC04+HEM3 to be used in yeast (Table 1). The constructed plasmid plYC04+HEM3+mCherry-UnaG (Table 1) was transformed into different deletion strains to validate the impact of introduced mutations on bilirubin formation (FIG. 9A). The wild-type strain carrying both hemoglobin A and mCherry-UnaG expression plasmids had the highest fluorescence yield, the introduction of rox1 deletion reduced the fluorescence yield by ˜30% (FIG. 9B3). The rox1 strain has more anaerobic nature, and lower bilirubin formation could be due to lower expression of heme degrading enzymes. The subsequent vps10 deletion reduced the fluorescence further by ˜40% (FIG. 9B4). The deletion of heme oxygenase encoding gene (HMX1, which is involved in biliverdin formation, the precursor of bilirubin) in rox1vps10 background did reduce the fluorescence by ˜10%, on the other hand, the deletion of PEP4 gene in the same strain background resulted in the 16% increase in bilirubin at the end of fermentation (FIG. 9B5 and FIG. 9B6 correspondingly). The bilirubin biosensor fluorescence was lower upon the expression of Hb fusion construct, which is more stable in tetramer form, compared to HbA (FIG. 10).

Strains with Higher Hemoglobin Production Exhibit Larger Size and Cell Density

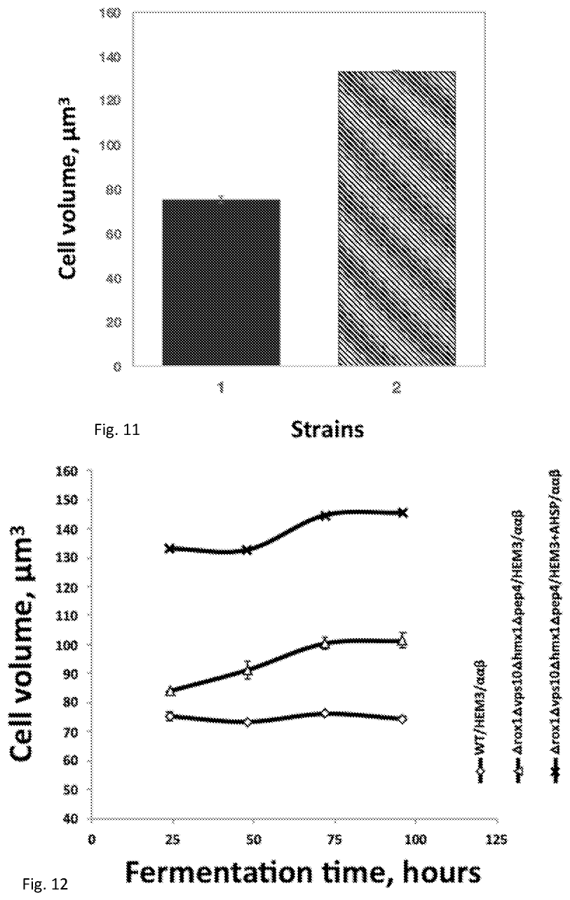

The AHSP strain (Δrox1Δvps10Δhmx1Δpep4/HEM3+AHSP/ααβ) with increased hemoglobin production increased its cell size ˜2-fold (FIG. 11). Larger cells usually contain more proteins. Under forced high protein production there is a constrain on the ribosomes activity and thus yeast adapts by slowing down the growth rate and increasing the cell size (Kafri M, Metzl-Raz E, Jona G et al. The Cost of Protein Production. Cell Rep. 2016. 14 (1): 22-31.). Interestingly, while the AHSP strain had the biggest cell volume (FIG. 11; FIG. 12), the cell volume of intermediate strain, Δrox1Δvps10Δhmx1Δpep4/HEM3/ααβ (it produced 58% lower Hb amount than AHSP (FIG. 3)), was only from 10% to 36% higher than control strain, WT/HEM3/ααβ (FIG. 12).

### Hemoglobin-GFP Construct is Localized to Cytoplasm

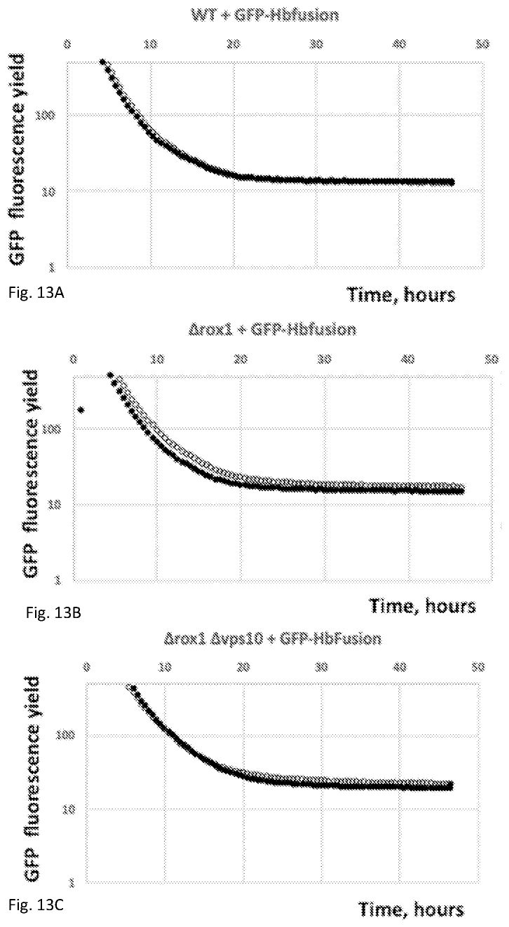

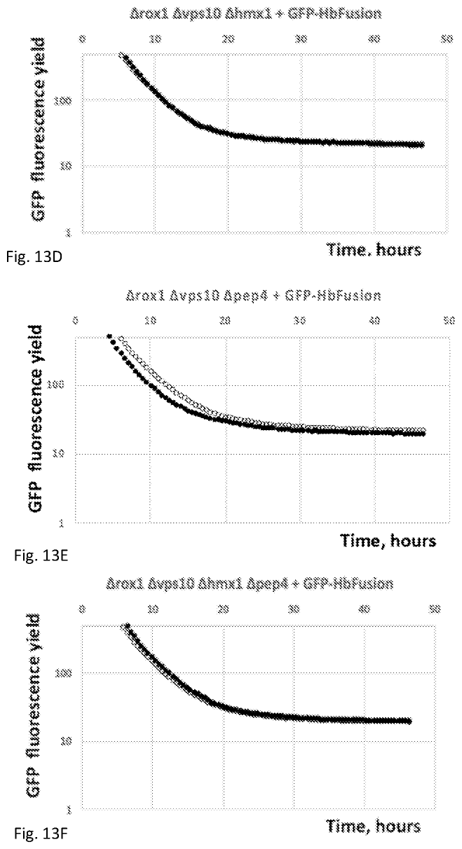

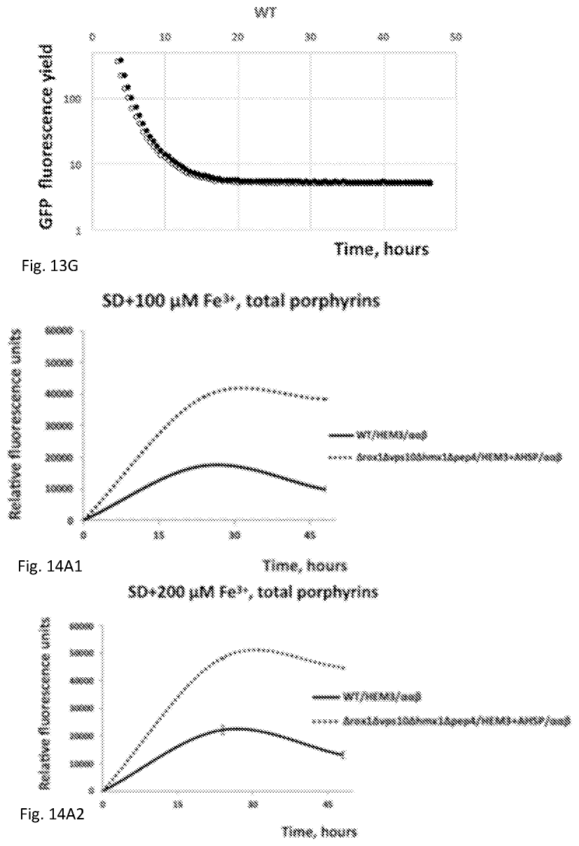

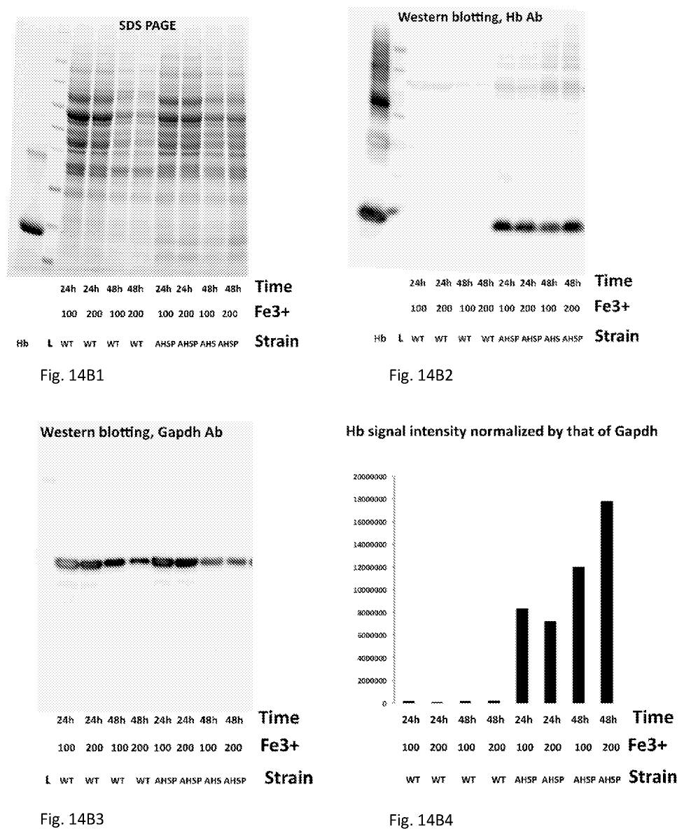

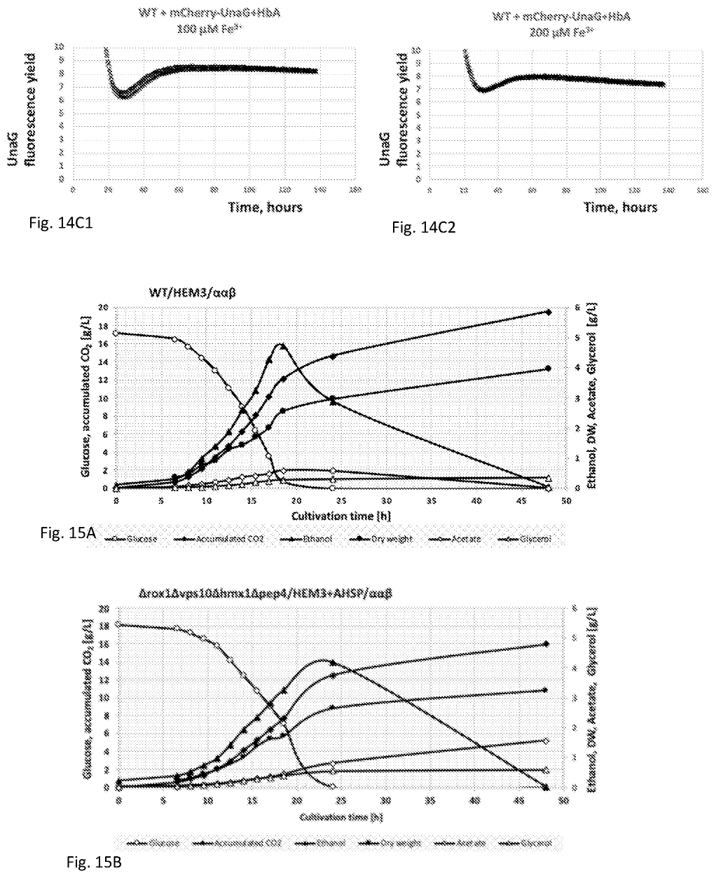

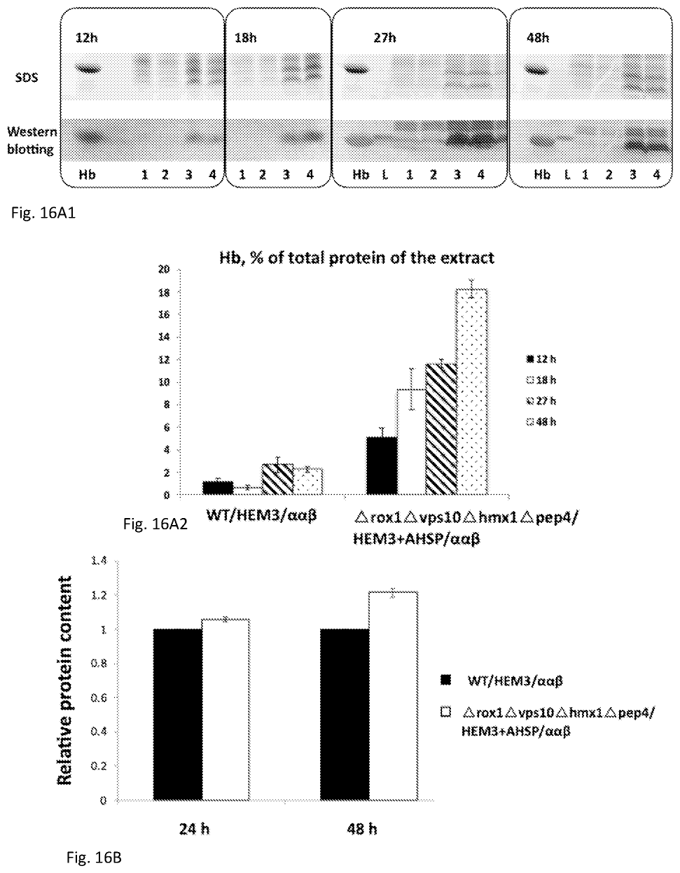

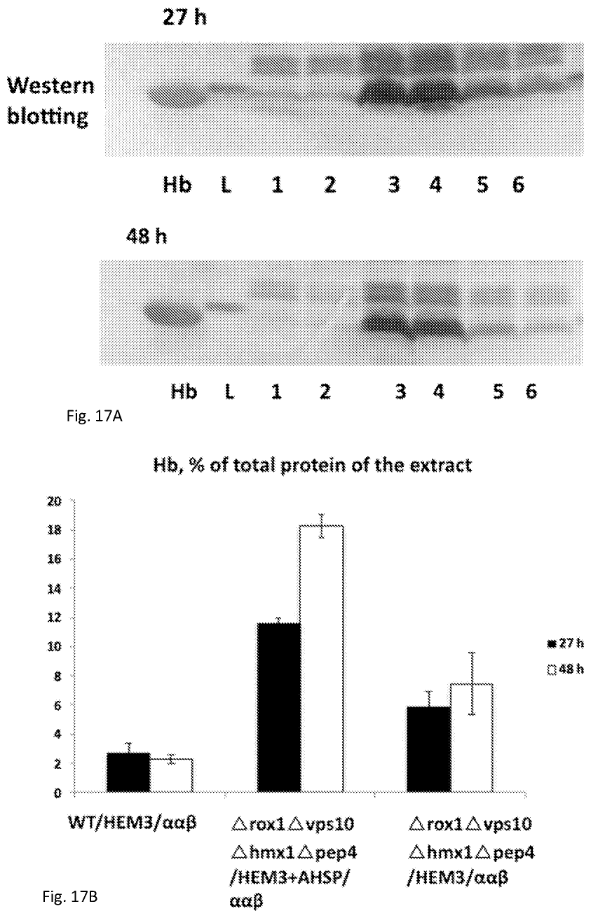

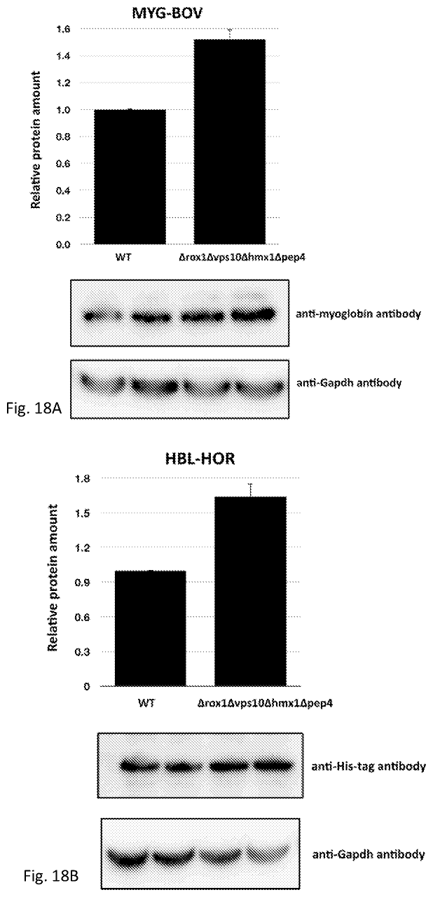

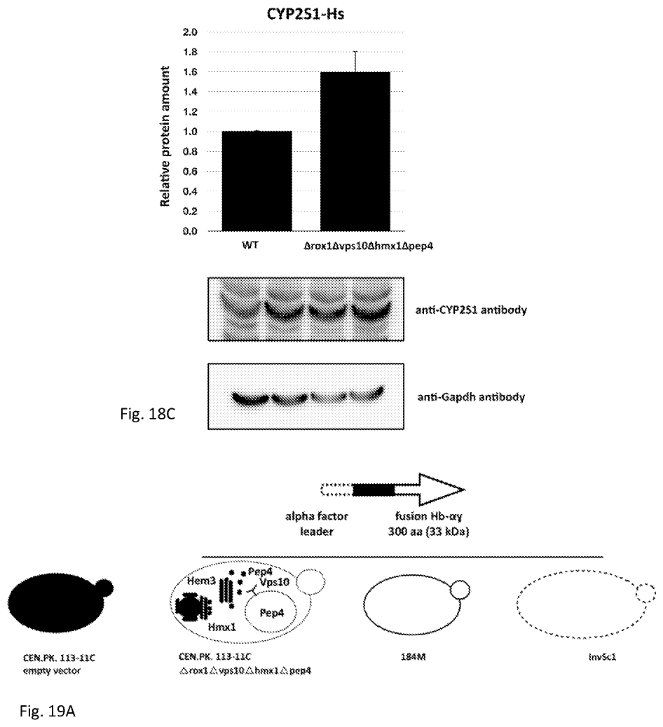

To monitor the hemoglobin expression and localization in constructed strains we introduced the reporter construct consisting of N-terminal GFP-Hb fusion (av) construct (“Materials and methods”). The GFP fluorescence yield (normalized by biomass) was increased substantially with the introduction of Δrox1, Δvps10, and Δhmx1 mutations compared to the wild-type (FIG. 13). The pep4 mutation increased the GFP fluorescence yield slightly further (FIG. 13).

### Supplementing Iron to the Medium Increases Recombinant Hemoglobin Production and Reduces the Bilirubin Formation

The final step of heme biosynthesis in S. cerevisiae is the incorporation of iron into the porphyrin ring by a ferrochelatase encoded by the HEM15 gene (Labbe-Bois R. The ferrochelatase from Saccharomyces cerevisiae. Sequence, disruption, and expression of its structural gene HEM15. J Biol Chem. 1990. 265 (13): 7278-7283.). The iron atom confers the stability to heme molecule, and heme degradation undergoes via release of iron, CO, and biliverdin by heme oxygenase.

We found that an increased amount of iron in the medium resulted in elevated level of porphyrin synthesis and hemoglobin protein production (FIG. 14A1, FIG. 14A2). In addition, the increased concentration of Fe³⁺ in the medium lead to lower amount of bilirubin formation (FIG. 14C1, FIG. 14C2).

### Hemoglobin Production in Bioreactors

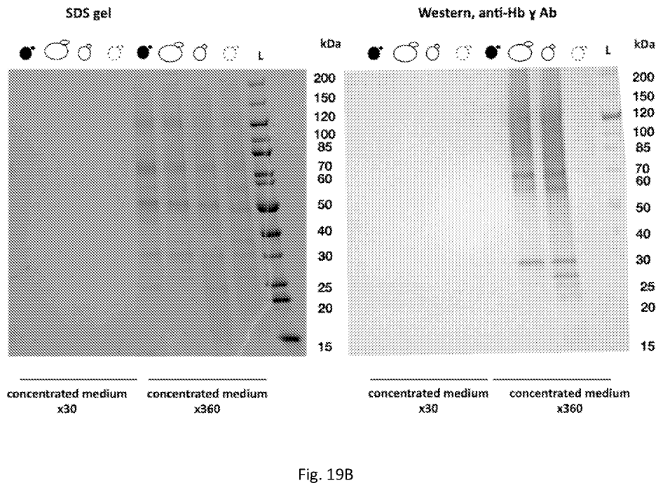

The hemoglobin production in constructed strains was studied in batch bioreactors with controlled aeration and pH (“Materials and Methods”). Under these conditions, the maximum specific growth rate of AHSP strain was 20% lower than that of the control strain, WT/HEM3/ααβ (Table 3). The AHSP strain produced higher yields of glycerol (2-fold higher), and acetate (30% higher), and increased oxygen consumption rate (FIG. 15; Table 3). On the other hand, the CO₂ yield was reduced by 6% (FIG. 15; Table 3). Increased glycerol, acetate production, and oxygen consumption rate indicate that the cell is coping with the redox imbalance in the cell. Under these conditions, the AHSP strain was found to produce hemoglobin up to 18% of its total protein content after 48 hours of cultivation, as estimated by Western blot (FIG. 16A1, FIG. 16A2), this level was 6-fold higher than that in the control strain, WT/HEM3/ααβ. The level of hemoglobin production in AHSP strain had positive correlation with total cellular protein increase at 48 hours of fermentation (FIG. 16B), which was probably due to the engineering of reduced vacuolar protein degradation. The intermediate strain, Δrox1Δvps10Δhmx14pep4 lacking AHSP protein, produced only ˜8% of Hb of its total protein content (FIG. 17), while the control strain, WT/HEM3/ααβ, accumulated up to ˜3-4% of intracellular hemoglobin, as reported before (Liu L, Martínez J L, Liu Z et al. Balanced globin protein expression and heme biosynthesis improve production of human hemoglobin in Saccharomyces cerevisiae. Metab Eng. 2014. 21:9-16.). In addition to hemoglobin, Δrox14vps10Δhmx14pep4 strain was also found to produce more other heme-containing proteins, such as human cytochrome P450 2S1, bovine myoglobin and non-symbiotic hemoglobin from barley (FIG. 18).

TABLE 3
Growth and metabolites yield of hemoglobin production strains during glucose fermentation in bioreactors.

| Strain | μₘₐₓ | Y_{SC} | SOUR | Y_{SE} | Y_{SX} | Y_{SA} | Y_{SG} | C-Balance |
|---|---|---|---|---|---|---|---|---|
| WT/H3/ ααβ | 0.24 ± 0.050 | 0.73 ± 0.010 | 5.14 ± 0.24 | 0.28 ± 0.008 | 0.13 ± 0.010 | 0.03 ± 0.01 | 0.02 ± 0.002 | 1.06 ± 0.02 |
| Δrox1Δvps10Δhmx1Δpep4/ | 0.24 ± 0.004 | 0.71 ± 0.010 | 6.06 ± 1.28 | 0.28 ± 0.010 | 0.14 ± 0.009 | 0.03 ± 0.001 | 0.02 ± 0.003 | 1.06 ± 0.02 |
| HEM3/ααβ | | | | | | | | |
| Δrox1Δvps10Δhmx1Δpep4/ | 0.20 ± 0.001 | 0.69 ± 0.010 | 5.89 ± 0.26 | 0.28 ± 0.008 | 0.14 ± 0.008 | 0.04 ± 0.0004 | 0.04 ± 0.004 | 1.07 ± 0.03 |
| HEM3 + AHSP/ααβ | | | | | | | | |

Maximum specific growth rate μₘₐₓ (h⁻¹), specific oxygen uptake rate (SOUR), yield of CO2 (Y_{SC}), yield of ethanol (Y_{SE}), yield of biomass (Y_{SX}), yield of acetate (Y_{SA}), and yield of glycerol (Y_{SG}), C-Balance - carbon balance. Four replicates were used in the experiment.

### Reducing Hemoglobin Degradation is Beneficial for its Secretion

The α-factor leader-hemoglobin fusion construct was expressed in three different strain backgrounds: INVSc1 (diploid strain developed for protein expression, Invirogen™), 184M (Huang M, Bai Y, Sjostrom S L et al. Microfluidic screening and whole-genome sequencing identifies mutations associated with improved protein secretion by yeast. Proc Natl Acad Sci USA. 2015. 112 (34): E4689-96.), and CENPK113-11c Δrox1 Δvps10Δhmx1Δpep4 constructed in this study (FIG. 19A). No hemoglobin was detected in the media prior medium concentration. However, after the concentrating the media by Amicon 10 kDa centrifugation filters (Merck Millipore) it was possible to detect the hemoglobin by Western blotting in two strains, 184M and CENPK113-11c Δrox1 Δvps10Δhmx1Δpep4 (FIG. 19B). No hemoglobin was detected in INVSc1 (FIG. 19B). While the protein band of ˜30 kDa was detected in both strains, the 184M strain had few bands of smaller size were detected (FIG. 19B). This could indicate a partial degradation of hemoglobin in 184M strain. Although the 184M strain carries several mutations including those that cause down-regulation of both ROX1 and VPS10 genes, but the PEP4 gene expression was not changed (Huang M, Bao J, Hallström B M et al. Efficient protein production by yeast requires global tuning of metabolism. Nat Commun. 2017. 8 (1): 1131.).

The modified yeast cell genotype may be for example: Δrox1Δvps10Δhmx1Δpep4/HEM3+alpha leader-Hbfusion carrying above mentioned genetic modifications, deletion of genes (ROX1, VPS10, HMX1, PEP4) and overexpression of the genes (HEM3, hemoglobin fusion protein (Hbfusion) comprising subunit alpha (a) and subunit gamma (γ) with alpha leader sequence of mating pheromone alpha factor (MF (ALPHA) 1) to direct hemoglobin into secretory pathway and outside of the cell into secretion medium). The nucleotide sequence of the secreted form of hemoglobin may be at least 95% identical with SEQ ID No. 13. The polypeptide sequence of the secreted form of hemoglobin may be at least 95% identical with SEQ ID No. 14, or the polypeptide has hemoglobin activity in yeast secretory pathway.

### Discussion

Human hemoglobins (Hbs) are heterotetramers of different subunits, depending on which developmental stage they are synthesized. All hemoglobins contain a prosthetic group (heme b), which is incorporated into globin chains co-transnationally, affecting the polypeptide folding. Heme availability is not only crucial for hemoglobin synthesis but also for its major function, since a heme-less hemoglobin will be unable to bind oxygen. The metabolic engineering can substantially increase heme production in yeast. The overexpression of the limiting gene of heme biosynthesis (Hoffman M, Góra M, Rytka J. Identification of rate-limiting steps in yeast heme biosynthesis. Biochem Biophys Res Commun. 2003. 310 (4): 1247-1253.), such as HEM3, which encodes the porphobilinogen deaminase, significantly increased both heme and hemoglobin production in S. cerevisiae (Liu L, Martínez J L, Liu Z et al. Balanced globin protein expression and heme biosynthesis improve production of human hemoglobin in Saccharomyces cerevisiae. Metab Eng. 2014. 21:9-16.).

In our strain design, i.e. our design of a genetically modified yeast cell in accordance with the present invention, we also used the HEM3 gene overexpression strategy. In yeast, the intracellular level of heme is tightly regulated at transcriptional level in response to the oxygen availability by the transcription factor Hap1. Hap1 activates the Rox1, a repressor of HEM13 gene (Keng T. HAP1 and ROX1 form a regulatory pathway in the repression of HEM13 transcription in Saccharomyces cerevisiae. Mol Cell Biol. 1992. 12 (6): 2616-2623; Martínez J L, Liu L, Petranovic D et al. Engineering the oxygen sensing regulation results in an enhanced recombinant human hemoglobin production by Saccharomyces cerevisiae. Biotechnol Bioeng. 2015. 112 (1): 181-188.). The deletion of the ROX1 gene also activates hypoxia-induced genes (Ter Linde J J, Steensma H Y. A microarray-assisted screen for potential Hap1 and Rox1 target genes in Saccharomyces cerevisiae. Yeast. 2002. 19 (10): 825-840.), and was previously shown to improve production of other heterologous proteins in S. cerevisiae, such as α-amylase, insulin, and invertase (Liu L, Zhang Y, Liu Z et al. Improving heterologous protein secretion at aerobic conditions by activating hypoxia-induced genes in Saccharomyces cerevisiae. FEMS Yeast Res. 2015. 15 (7). pii: fov070.). To eliminate the HEM13 gene inhibition by Rox1 we used Δrox1 as the background strain for hemoglobin expression. The Δrox1 strain proved to produce higher porphyrin amounts under HEM3 gene overexpression. Heterologous protein synthesis is negatively affected by the homologous protein degradation mechanisms in the host and the success of protein production greatly depends on the suppression of these processes. To address this, we engineered a hemoglobin-producing S. cerevisiae strain, i.e. a genetically modified yeast cell in accordance with the present invention, with reduced degradation of heme and reduced degradation of globin peptides. The intracellular level of heme is regulated by heme degradation. The heme oxygenase encoded by the HMX1 gene degrades heme upon iron starvation and oxidative stress (Protchenko O, Philpott C C. Regulation of intracellular heme levels by HMX1, a homologue of heme oxygenase, in Saccharomyces cerevisiae. J Biol Chem. 2003. 278 (38): 36582-7.). Under conditions of high protein production misfolded proteins are targeted to the vacuole degradation. The VPS10 gene, encoding type I transmembrane sorting receptor for multiple vacuolar hydrolases, is involved in vacuolar targeting of unfolded proteins (Marcusson E G, Horazdovsky B F, Cereghino J L et al. The sorting receptor for yeast vacuolar carboxypeptidase Y is encoded by the VPS10 gene. Cell. 1994. 77 (4): 579-586.). The deletion of the VPS10 gene and its decreased activity was shown to improve the production of other heterologous proteins (Hong E, Davidson A R, Kaiser C A. A pathway for targeting soluble misfolded proteins to the yeast vacuole. J Cell Biol. 1996. 135 (3): 623-633; Huang M, Bao J, Hallström B M et al. Efficient protein production by yeast requires global tuning of metabolism. Nat Commun. 2017. 8 (1): 1131.). The PEP4 gene encodes a vacuolar aspartyl protease, important for the recycling of damaged proteins by oxidative stress. The mutation of the PEP4 gene was beneficial for the production of different heterologous proteins in different yeast strains (Marques M, Mojzita D, Amorim M A et al. The Pep4p vacuolar proteinase contributes to the turnover of oxidized proteins but PEP4 overexpression is not sufficient to increase chronological lifespan in Saccharomyces cerevisiae. Microbiology. 2006. 152 (Pt 12): 3595-3605; and Wang Z Y, He X P, Zhang B R. Over-expression of GSH1 gene and disruption of PEP4 gene in self-cloning industrial brewer's yeast. Int J Food Microbiol. 2007. 119 (3): 192-199.).

When we combined ROX1, VPS10, HMX1, and PEP4 gene deletions, i.e. in a genetically modified yeast cell in accordance with the present invention, we were able to detect hemoglobin in the protein gels even without Western blotting. By using bilirubin-binding fluorescent biosensor, we confirmed that this mutant, i.e. a genetically modified yeast cell in accordance with the present invention, produced much lower amount of hemoglobin degradation product, bilirubin, and thus more product. The supplementation of iron also improved the hemoglobin yield and reduced the bilirubin formation in our strains. The heme oxygenase expression is regulated by iron oxidative stress (Protchenko O, Philpott C C. Regulation of intracellular heme levels by HMX1, a homologue of heme oxygenase, in Saccharomyces cerevisiae. J Biol Chem. 2003. 278 (38): 36582-7; and Collinson E J, Wimmer-Kleikamp S, Gerega S K et al. The yeast homolog of heme oxygenase-1 affords cellular antioxidant protection via the transcriptional regulation of known antioxidant genes. J Biol Chem. 2011. 286 (3): 2205-2214.) and thus its expression is lower in the medium with higher iron amount and in Δrox1 strain. The overexpression of the human AHSP gene (α-hemoglobin stabilizing protein), which stabilizes the hemoglobin molecule and prevents its degradation and oxidation in erythrocytes (Kihm A J, Kong Y, Hong W et al. An abundant erythroid protein that stabilizes free alpha-haemoglobin. Nature. 2002. 417 (6890): 758-763; Feng L, Gell D A, Zhou S et al. Molecular mechanism of AHSP-mediated stabilization of alpha-hemoglobin. Cell. 2004. 119 (5): 629-640; Yu X, Kong Y, Dore L C et al. An Erythroid Chaperone That Facilitates Folding of Alpha-Globin Subunits for Hemoglobin Synthesis J Clin Invest. 2007. 117 (7): 1856-1865; Mollan T L, Yu X, Weiss M J et al. The role of alpha-hemoglobin stabilizing protein in redox chemistry, denaturation, and hemoglobin assembly. Antioxid Redox Signal. 2010. 12 (2): 219-231.), resulted in 58% increase of hemoglobin production in Δrox1Δvps10Δhmx14pep4 yeast strain. The AHSP expression reduced the ROS formation at the early stage of fermentation (at 6 hours), however at 24 hours of fermentation AHSP strain accumulated the highest amount of ROS, which had positive correlation with amount of hemoglobin and cellular porphyrins. In bioreactors, AHSP strain hemoglobin level was 18% with respect to the total intracellular protein content. This was 2.6 times higher than that reported before by Martínez J L, Liu L, Petranovic D et al. Engineering the oxygen sensing regulation results in an enhanced recombinant human hemoglobin production by Saccharomyces cerevisiae. Biotechnol Bioeng. 2015. 112 (1): 181-188. High protein production, which was achieved by engineering the reduced protein degradation, caused the cell to increase its volume accompanied with increase of its total protein content (˜20%), and to reduce the growth rate by 313% in AHSP strain. These are the features of the cell adapting to high protein synthesis burden and were reported before for using reporter constructs in S. cerevisiae (Kafri M, Metzl-Raz E, Jona G, et al. The Cost of Protein Production. Cell Rep. 2016. 14 (1): 22-31.).

High level of intracellular hemoglobin production caused changes in the fermentation profile of AHSP strain. Increased oxygen consumption, production of by-products glycerol and acetate by this strain indicate the redox imbalance. The complex phenotype of constructed strain as combination of rox1 (leading to hypoxic genes expression), hmx1 (leading to iron depletion) mutations and hemoglobin production (depleting iron) caused overall oxygen limitation in the cell. Glycerol and acetate are produced to satisfy NADH/NADPH balance under anaerobic conditions (Villadsen J, Nielsen J, Liden G. Bioreaction Engineering Principles. Springer US. 2011.). When cells are deprived of iron, their respiratory chain does not function well and they accumulate NADH, and they respond by inducing the expression of the GPD2 gene and producing glycerol (Ansell R, Adler L. The effect of iron limitation on glycerol production and expression of the isogenes for NAD (+)-dependent glycerol 3-phosphate dehydrogenase in Saccharomyces cerevisiae. FEBS Lett. 1999. 461 (3): 173-177.). The strategy of addressing the cofactor imbalance in this strain by metabolic engineering could be used to improve the hemoglobin production further.

In conclusion, we engineered a S. cerevisiae strain, i.e. a genetically modified yeast cell in accordance with the present invention, that is capable of producing up to 18% (of total cell protein) of human hemoglobin (HbA) in relation to the total cell protein. This strain, i.e. a genetically modified yeast cell in accordance with the present invention, can be used as sustainable and safe source of hemoglobin for the development of hemoglobin-based oxygen carriers (HBOCs), or other heme-containing proteins (e.g. for sustainable food or feed production), or heme enzymes (e.g. P450). As the strain described herein is able to produce hemoglobin and other heme proteins as P450 in high yield from glucose it can be used as a hemoglobin or other heme proteins producer for industries developing HBOCs or food products.

### Materials and Methods

### Media and Strains Growth Conditions

Media and strains used in experiments for embodiment of the invention are provided below. Strains used in this study are listed in Table 2 above. The strains of Saccharomyces cerevisiae CEN.PK 113-11C (MATa his341 ura 3-52 MAL2-8c SUC2) (Entian and Kotter, 1998.) and its Δrox1 mutant (Liu L, Zhang Y, Liu Z et al. Improving heterologous protein secretion at aerobic conditions by activating hypoxia-induced genes in Saccharomyces cerevisiae. FEMS Yeast Res. 2015. 15 (7). pii: fov070.) were used as hosts for human hemoglobin production. Yeast strains were maintained at 30° C. in a complete rich medium YPD (5 g/L yeast extract, 10 g/L peptone, 20 g/L glucose). Transformants with hemoglobin A expression plasmids plYC04+HEM3 and pSP-GM1+ααβ (Liu L, Martínez J L, Liu Z et al. Balanced globin protein expression and heme biosynthesis improve production of human hemoglobin in Saccharomyces cerevisiae. Metab Eng. 2014. 21:9-16.) were selected on synthetic complete medium SD without both uracil and histidine (6.9 g/L yeast nitrogen base with ammonium sulphate w/o amino acids (Formedium™), 0.75 g/L synthetic complete drop-out mixture w/o histidine and uracil (Formedium™), pH 6.0) containing 20 g/L glucose as carbon source. Additional iron was added to the SD medium (SD with Fe³⁺) (100 μM of ferric citrate, Sigma-Aldrich). Deletion mutants were selected on YPD medium with G418 at the concentration of 0.2 g/L. For kanMX marker removal by Cre recombinase induction, transformants were grown on YPG medium (5 g/L yeast extract, 10 g/L peptone, 10 g/L galactose) overnight. For the evaluation of porphyrin production in Δrox1 strain, 5-aminolevulinic acid (5-ALA) was added in SD media at the concentration of 1 mM.

### Generation of Gene Knockout Strains

Oligonucleotide primers and plasmids used in this study are listed in Table 1. The deletion cassettes with the dominant selection marker kanMX expressed under control of Ashbya gossypii TEF1 (Steiner S, Philippsen P. Sequence and promoter analysis of the highly expressed TEF gene of the filamentous fungus Ashbya gossypii. Mol Gen Genet. 1994. 242 (3): 263-271.) were used for the gene knockouts. The subsequent marker removal was done by the Cre-lox system (Cre recombinase was expressed under promoter GAL1 of S. cerevisiae) (Wenning L, Yu T, David F et al. Establishing very long-chain fatty alcohol and wax ester biosynthesis in Saccharomyces cerevisiae. Biotechnol Bioeng. 2017. 114 (5): 1025-1035.). The deletion cassettes carried kanMX and Cre-recombinase flanked with LoxP and ˜50 bp of nucleotide sequences homologous to HMX1, VPS10, and PEP4 target genes of S. cerevisiae. Each deletion cassette was amplified in 2 fragments from template plasmids pDel1 and pDel2 (Table 1, (Wenning L, Yu T, David F et al. Establishing very long-chain fatty alcohol and wax ester biosynthesis in Saccharomyces cerevisiae. Biotechnol Bioeng. 2017. 114 (5): 1025-1035.)), containing 335 bp overlapping region of kanMX gene to be repaired in vivo in yeast after the transformation (fragment 1: target gene 5′-sequence-loxP-half kanMX gene; fragment 2: kanMX gene (second half with overlap)-GAL-promoter-Cre recombinase-LoxP-target gene 3′ sequence, that were then co-transformed into Δrox1 mutant. The HMX1 gene deletion cassette was amplified by Del-HMX1-1 and Del1-rev primers pair (PCR fragment 1), Del2-for and Del2-HMX1-2 (PCR fragment 2). The VPS10 gene deletion cassette was amplified by VPS10-1 and Del1-rev (PCR fragment 1), Del2-for and VPS10-2 (PCR fragment 2). The PEP4 gene deletion cassette was amplified by PEP4-4 and Del1-rev (PCR fragment 1), Del2-for and PEP4-2 (PCR fragment 2) (Table 1). The transformants with deletion cassettes were selected on YPD medium with G418 at the concentration 0.2 g/L. The gene deletions were verified by PCR analysis and obtained mutants were selected for further studies. To induce the Cre recombinase expression, the transformants were grown overnight in rich medium with galactose (YPG) and then plated on YPD. The transformants, that lost the ability to grow on YPD with G418 after this treatment, were selected for further studies.

### Plasmids and Synthetic DNA

Plasmids constructed in this study and oligonucleotides used are listed in Table 1. The sequence of human alpha hemoglobin stabilizing protein (AHSP) gene was codon-optimized for S. cerevisiae (https://www.kazusa.or.jp/codon/cgi-bin/showcodon.cgi?species=4932) and obtained as synthetic DNA from GenScript. The codon-optimized fragment was then amplified with AHSP-1 and AHSP-2 primers and cloned into the plasmid plYC04+HEM3 under promoter PGK1 resulting in the plasmid plYC04+HEM3+AHSP. Adapting the hemoglobin fusion (ay subunits fusion) construct for the bacterium E. coli (Chakane S. (2017). Towards New Generation of Hemoglobin-Based Blood Substitutes. Department of Chemistry, Lund University) for the use in yeast, we first codon-optimized it for S. cerevisiae expression (designated Hbfusion). The GFP ORF was amplified from the plasmid p416TEFGFP (Refer to Jensen E D, Ferreira R, Jakočiūnas T et al. Transcriptional reprogramming in yeast using dCas9 and combinatorial gRNA strategies. Microb Cell Fact. 2017. 16 (1): 46.), with primers HbF-GFP-1 and HbF-GFP-2, fused with Hbfusion construct amplified with primers HbF-GFP-3 and H3AFHb-2, and cloned into plYC04+HEM3 plasmid under strong constitutive promoter PGK1 using Gibson Assembly® (New England Biolabs, NEB) resulting into plasmid plYC04+HEM3+GFP-Hbfusion. plYC04+HEM3+mCherry-UnaG was constructed on the base of plYC04+HEM3 (Liu L, Martínez J L, Liu Z et al. Balanced globin protein expression and heme biosynthesis improve production of human hemoglobin in Saccharomyces cerevisiae. Metab Eng. 2014. 21:9-16.). The mCherry-UnaG fusion was amplified from mCherry-FDD vector (Addgene #80629 (Navarro R, Chen L C, Rakhit R et al. A Novel Destabilizing Domain Based on a Small-Molecule Dependent Fluorophore. ACS Chem Biol. 2016. 11 (8): 2101-4.)) with primers FDD-B/FDD-X (Table 1) and ligated with plYC04+HEM3 digested with BamHI and XhoI. CPOT+α-leader-Hbfusion+HEM3 was constructed on the base of CPOTud (Liu Z, Tyo K E, Martínez J L et al. Different expression systems for production of recombinant proteins in Saccharomyces cerevisiae. Biotechnol Bioeng. 2012. 109 (5): 1259-68.). The fragment carrying α-leader sequence was amplified with primers pairs Alpha-1 and Alpha-2. The fragment carrying hemoglobin αγ-fusion (Hbfusion) was amplified with primers Fusion-1 and Fusion-2 from plYC04+HEM3+GFP-Hbfusion. The obtained fragments were cloned into KpnI and NheI digested CPOT plasmid by Gibson Assembly® (New England Biolabs, NEB) resulting in the plasmid CPOT+α-leader-Hbfusion. The HEM3 gene under control of promoter TEF1 was amplified with primers pair HEM3CPOT-1 and HEM3CPOT-2 and cloned into BamHI site of CPOT+α-leader-Hbfusion. plYC04+HEM3+α-leader-Hbfusion was constructed on the basis of plYC04+HEM3 (Liu L, Martínez J L, Liu Z et al. Balanced globin protein expression and heme biosynthesis improve production of human hemoglobin in Saccharomyces cerevisiae. Metab Eng. 2014. 21:9-16.). The fragment carrying α-leader-Hbfusion construct was amplified from CPOT+α-leader-Hbfusion+HEM3 with primers H3AFHb-1 and H3AFHb-2 and cloned into BamHI and XhoI digested plYC04+HEM3. The pESC-URA+CYP2S1, where the CYP2S1 ORF was cloned under promoter GAL10 into BamHI and XhoI of pESC-URA and was obtained from GenScript as synthetic DNA with codon adaptation for S. cerevisiae expression and carried His6-tag. The pESC-URA+MYG-BOV, where the ORF of MB gene of Bos taurus was cloned under promoter GAL10 into BamHI and XhoI of pESC-URA and was obtained from GenScript as synthetic DNA with codon adaptation for S. cerevisiae expression and carried His6-tag. The pESC-URA+HBL-HOR, where the ORF of GLB1 gene of Hordeum vulgare was cloned under promoter GAL10 into BamHI and XhoI of pESC-URA and was obtained from GenScript as synthetic DNA with codon adaptation for S. cerevisiae expression and carried His6-tag.

### Glucose Fermentations and Metabolites Analysis

Batch glucose fermentations were performed in flasks and under strictly controlled conditions in bioreactors. The shake flask fermentations were performed at 30° C. in 25 ml of liquid medium at 200 rpm, inoculated with an initial OD600 of 0.2 from the pre-cultures. The batch fermentations were performed in 1.0 L Biostat Qplus© bioreactors (Sartorius Stedim Biotech, Germany) with a working volume of 500 ml. The temperature was maintained at 30° C. and pH at 6.0. Bioreactors were inoculated with an initial OD600 of 0.1 from the pre-cultures. The amount of dissolved oxygen was measured by oxygen sensors and maintained above 30%. The volumetric flow (aeration) was set to 60 L/h (2 vvm) and constant agitation stirrer speed at 600 rpm. The dry weight was measured by collecting the biomass on membrane filters (0.45 μm, MontaMil® MCE, Frisenette, Denmark) with subsequent drying. The metabolites in the cultivation media were measured in the cultivation media by HPLC (Dionex Ultimate 3000 HPLC (Model 1100-1200 Series HPLC System, Agilent Technologies, Germany) with HPX-87H column (BIO-RAD, USA)). The off-gas from the bioreactors was passed through a foam-trap and analyzed by a mass spectrometer (Model Prima PRO Process MS, Thermo Fisher Scientific™, United Kingdom).

### ROS Detection

The Reactive Oxygen Species (ROS) level was measured in vivo using the dihydrorhodamine 123 dye by the protocol described by Johansson M, Chen X, Milanova S et al. PUFA-induced cell death is mediated by Yca1p-dependent and -independent pathways, and is reduced by vitamin C in yeast. FEMS Yeast Res. 2016. 16 (2): fow007. For this purpose, the 6 h hours of fermentation, cells were collected by centrifugation and washed with 50 mM sodium citrate buffer. The cells were further incubated with 50 mM sodium citrate buffer supplemented with 50 μM dihydrorhodamine 123 for 30 min in the dark. After the staining, the cells were spun down and washed with 50 mM sodium citrate buffer. The formation of rodamine (oxidized form of dihydrorhodamine 123) was detected by fluorescence using the FLUOstar Omega microplate reader (with the excitation 485 nm and emission 520 nm filters) and Guava easyCyte™ 8HT flow cytometer (Millipore).

### Porphyrins Content Analysis

Cellular heme and porphyrin content were determined as described before (Liu L, Martínez J L, Liu Z et al. Balanced globin protein expression and heme biosynthesis improve production of human hemoglobin in Saccharomyces cerevisiae. Metab Eng. 2014. 21:9-16.). Free cellular heme and total porphyrin content was determined after oxalic acid treatment by their fluorescence with excitation at A=400 nm and emission at A=600 nm on a FLUOstar Omega plate reader spectrophotometer.

### Detection of Carboxyhemoglobin by Absorption Spectra

The yeast cells crude extracts were prepared as described earlier (Ishchuk O P, Martínez J L, Petranovic D. Improving the production of cofactor containing proteins: production of human hemoglobin in yeast. In: Gasser B and Mattanovich D (eds). Recombinant Protein Production in Yeast. Methods in Molecular Biology, vol. 1923. 2019. Humana Press, New York, NY.). 100 mM potassium phosphate buffer for cells crude extracts contained protease inhibitor cocktail (Fisher Scientific), CO-releasing compound CORM-3 (Sigma-Aldrich) at 0.6 mg/ml, 2 mM MgCl₂, 1 mM dithiothreitol and 1 mM EDTA. After cells debris removal, the carboxyhemoglobin amount was determined by spectra analysis of protein extracts of samples with the same concentration (13 mg/ml).

### Determination of Cell Volume

The yeast cell volume was determined using CASY Model TT Cell Counter and Analyzer (Roche Diagnostics International Ltd). Cells were collected from bioreactors at 24, 48, 72 and 96 h of cultivation, re-suspended in CASY ton buffer and analyzed using capillary of 60 μm.

### Protein Extraction and Western Blotting.

Total protein was extracted by TCA treatment as described in Baerends R J, Faber K N, Kram A M et al. A stretch of positively charged amino acids at the N terminus of Hansenula polymorpha Pex3p is involved in incorporation of the protein into the peroxisomal membrane. J Biol Chem. 2000. 275 (14): 9986-9995 and proteins separated by electrophoresis on precast SDS-polyacrylamide gels (4-20% gradient, Mini-PROTEAN® TGX Stain-Free™ Precast Gels, BIO-RAD), electro-transferred to PVDF membrane (Trans-Blot®Turbo Mini PVDF Transfer Packs, BIO-RAD) and hybridized with anti-hemoglobin antibodies (Hemoglobin α antibody (D-16): sc-31110, goat polyclonal, Santa Cruz Biotechnology). For hemoglobin signal detection, secondary antibodies were used conjugated with either alkaline phosphatase (Anti-goat IgG, Sigma-Aldrich) or horseradish peroxidase (Anti-goat IgG, Fisher-Scientific). The signal intensity was analyzed in Image Lab™ (BIO-RAD).

### Protein Concentration in Whole Cell

The protein content was determined as described by Verduyn C, Postma E, Scheffers W A et al. Physiology of Saccharomyces cerevisiae in anaerobic glucose-limited chemostat cultures. J Gen Microbiol. 1990. 136 (3): 395-403. Equal amount of yeast dry weight was used when analyzing each strain.

### Statistical Analysis

The software package Minitab® 18.1, were used to analyze the obtained data.

1. A genetically modified yeast cell, wherein the yeast cell comprises a genetic modification comprising overexpression of yeast gene encoding porphobilinogen deaminase (HEM3), the HEM3 gene having at least 80% identity with SEQ ID No. 7, characterized in that the genome of the modified yeast cell further comprises one or more genetic modifications in one or more genes selected from: genes coding for heme-dependent repressor of hypoxic genes (ROX1), genes coding for heme oxygenase (HMX1), genes coding for a receptor for vacuolar proteases (VPS10), and genes coding for vacuolar proteinase (PEP4), the one or more genetic modifications being such that expression of a polypeptide from such a gene is reduced or disrupted or the polypeptide expressed is non-functional.

2. The genetically modified yeast cell of claim 1, wherein the yeast genome of the modified yeast cell comprises one or more genetic modifications in the genes coding for a heme-dependent repressor of hypoxic genes (ROX1).

3. The genetically modified yeast cell of claim 1, wherein the yeast genome of the modified yeast cell comprises one or more genetic modifications in the genes coding for a receptor for vacuolar proteases (VPS10).

4. The genetically modified yeast cell of claim 1, wherein the yeast genome of the modified yeast cell comprises one or more genetic modifications in the genes coding for heme oxygenase (HMX1).

5. The genetically modified yeast cell of claim 1, wherein the yeast genome of the modified yeast cell comprises one or more genetic modifications in the genes coding for vacuolar proteinase A (PEP4).

6. The genetically modified yeast cell of claim 1, wherein the yeast cell comprises a human gene encoding erythroid molecular chaperone (AHSP), the AHSP gene having at least 80% identity with SEQ ID No. 5, and wherein the AHSP gene is overexpressed.

7. The genetically modified yeast cell of claim 1, wherein the genetically modified yeast cell comprises genes coding for human hemoglobin or genes coding for non-human hemoglobins, wherein the non-human hemoglobins contain heme as a cofactor and a globin part that reversibly binds gaseous ligands.

8. The genetically modified yeast cell of claim 1, wherein the genetically modified yeast cell comprises genes coding for hemoglobin-based oxygen carriers (HBOCs), myoglobin or P450 enzymes.

9. The genetically modified yeast cell of claim 1, wherein the yeast cell is Saccharomyces cerevisiae, Pichia pastoris, Hansenula polymorpha or Yarrowia lipolytica.

10. The genetically modified yeast cell of claim 1, wherein the yeast genome of the modified yeast cell comprises one or more genetic modification in the genes coding for VPS10 and ROX1.

11. The genetically modified yeast cell of claim 1, wherein the yeast genome of the modified yeast cell comprises one or more genetic modification in the genes coding for VPS10, ROX1 and HMX1.

12. The genetically modified yeast cell of claim 1, wherein the yeast genome of the modified yeast cell comprises one or more genetic modification in the genes coding for VPS10, ROX1, HMX1 and PEP4.

13. The genetically modified yeast cell of claim 12, wherein the yeast cell further comprises a human gene encoding erythroid molecular chaperone (AHSP), the AHSP gene having at least 80% identity with SEQ ID No. 5, and wherein the AHSP gene is overexpressed.

14. The genetically modified yeast cell of claim 13, wherein the genetically modified yeast cell further comprises genes coding for human hemoglobin or genes coding for non-human hemoglobins, wherein the non-human hemoglobins contain heme as a cofactor and a globin part that reversibly binds gaseous ligands.

15. The genetically modified yeast of claim 1, wherein the genetically modified yeast comprises in its genome 1, 2, 3, 4 or more copies of genes encoding HEM3; multi-copy of plasmids encoding HEM3; or the HEM3 gene is modified to comprise a strong constitutive promoter, optionally the strong constitutive promoter is from the genes TEF1 or PGK1.

16. The genetically modified yeast of claim 1, wherein the one or more genetic modifications comprise deletion of the open reading frame of the ROX1 gene, deletion of the open reading frame of the VPS10 gene, deletion of the open reading frame of the HMX1 gene and/or deletion of the open reading frame of the PEP4 gene.

17. The genetically modified yeast of claim 1, wherein the genetically modified yeast comprises in its genome 1, 2, 3, 4 or more copies of genes encoding AHSP; multi-copy of plasmids encoding AHSP; or the AHSP gene is modified to comprise a strong constitutive promoter, optionally the strong constitutive promoter is from the genes TEF1 or PGK1.

18. The genetically modified yeast of claim 7, wherein the genetically modified yeast comprises in its genome 1, 2, 3, 4 or more copies of genes encoding human hemoglobin or non-human hemoglobins; multi-copy of plasmids encoding human hemoglobin or non-human hemoglobins; or the human hemoglobin gene or non-human hemoglobins is/are modified to comprise a strong constitutive promoter, optionally the strong constitutive promoter is from the gene TEF1 or PGK1.

19. The genetically modified yeast cell of claim 1, wherein the genetically modified yeast cells comprise a copy of the human HBA gene cloned under the strong promoter PGK1, a copy of the human HBA gene cloned under the strong promoter TEF1, and/or a copy of the human HBB gene cloned under the strong promoter PGK1.

20. The genetically modified yeast cell according to claim 1, wherein the yeast cell is Saccharomyces cerevisae.
